(19) United States
(12) Patent Application Publication
(10) Pub. No.: US 2017/0058264 A1
(43) Pub. Date: Mar. 2, 2017

(54) FORMATION OF MULTICELLULAR TUMOROIDS AND USES THEREOF

(71) Applicants: UNIVERSITY OF SOUTH FLORIDA, Tampa, FL (US); TRANSGENEX THERAPEUTICS, LLC, Tampa, FL (US)

(72) Inventors: Subhra Mohapatra, Lutz, FL (US); Shyam S. Mohapatra, Lutz, FL (US); Mahasweta Das, Wesley Chapel, FL (US)

(73) Assignees: University of south Florida, Tampa, FL (US); TRANSGENEX THERAPEUTICS, LLC, Tampa, FL (US)

(21) Appl. No.: 14/787,648

(22) PCT Filed: May 19, 2015
(86) PCT No.: PCT/US15/31571

§ 371 Date: Oct. 28, 2015

Related U.S. Application Data
(60) Provisional application No. 62/000,081, filed on May 19, 2014.

(51) Int. Cl. C12N 5/09

(52) U.S. Cl. CPC C12N 5/0693; C12N 2502/09; C12N 2502/095; C12N 2502/1323; C12N 2502/1358; C12N 2503/02; C12N 2513/00

(57) ABSTRACT

Described herein are compositions and methods of forming multi-cellular tumoroids. Also described herein are methods of using the multi-cellular tumoroids.

### CROSS-REFERENCE TO RELATED APPLICATIONS

This application claims the benefit of U.S. Provisional Application Ser. No. 62/000,081 filed on May 19, 2014, having the title “A Method of Anti-Cancer Drug Discover”, the entirety of which is incorporated herein by reference.

### STATEMENT REGARDING FEDERALLY SPONSORED RESEARCH OR DEVELOPMENT

This invention was made with government support under grant number HHSN261201300044C awarded by the National Institute of Health. The government has certain rights to this invention.

### BACKGROUND

Potential anti-cancer drugs entering clinical development have a high level of attrition (about 95%) despite the rising cost of new drug development (about 800 million). Such a high rate of attrition has been attributed to the current approaches used for anti-cancer drug discovery, efficacy testing, and drug development in two-dimensional (2D) cell-culture assays and in vivo animal models. As such, there is an urgent and unmet need for improved tools and techniques for anti-cancer drug discovery, efficacy testing, and drug development.

### SUMMARY

Described herein are methods of forming multi-cellular tumoroids. In some aspects, the methods can contain the step of co-culturing tumor cells (TCs), cancer associated fibroblast cells (CAFs), and epithelial cells (ECs) in a cell culture media, where the ratio of the number of TCs to CAFs to ECs is 5 to 1 to 1, and where the cell culture media comprises an amount of a mesenchymal stem cell conditioned media. The tumor cells can be breast cancer cells. The tumor cells can be derived from a subject. In some embodiments, the breast cancer cells are derived from a subject's tumor. In some aspects, the mesenchymal stem cell conditioned media is present at least at about 20% of the total amount of the cell culture media. In further aspects, the mesenchymal stem cell conditioned media is present at least at about 20% to about 50% of the total amount of the cell culture media. The mesenchymal stem cell conditioned media can contain at least about 800 pg/mL VEGF. The mesenchymal stem cell conditioned media can contain at least about 100 pg/mL IL-6. the mesenchymal stem cell conditioned media the mesenchymal stem cell conditioned media contains at least about 1200 pg/mL TGF-β1. at least about 1200 pg/mL TGF-β1. The mesenchymal stem cell conditioned media can contain at least about 800 pg/mL VEGF, at least about 100 pg/mL IL-6, and at least about 1200 pg/mL TGF-β1. In additional aspects, the cell culture media can further contain about % to about 10% Matrigel. In some aspects, the TCs, CAFs, and ECs are co-cultured on a three-dimensional scaffold. The three-dimensional scaffold can be a fibrous induced smart scaffold.

Also described are methods of determining the efficacy of an anti-cancer drug containing the steps of forming a multicellular tumoroid, where forming the multicellular tumoroid contains the step of co-culturing tumor cells (TCs), cancer associated fibroblast cells (CAFs), and epithelial cells (ECs) in a cell culture media, where the ratio of the number of TCs to CAFs to ECs is 5 to 1 to 1, and where the cell culture media comprises an amount of a mesenchymal stem cell conditioned media; and exposing the multicellular tumoroid to an amount of the anti-cancer drug. In some aspects, the tumor cells are breast cancer cells. The breast cancer cells can be derived from a subject's tumor. The breast cancer cells can be from a standard breast cancer cell line. The mesenchymal stem cell conditioned media can be present at least at about 20% of the total amount of the cell culture media. The mesenchymal stem cell conditioned media can be present at least at about 20% to about 50% of the total amount of the cell culture media. The mesenchymal stem cell conditioned media can contain at least about 800 pg/mL VEGF, at least about 100 pg/mL IL-6, and at least about 1200 pg/mL TGF-β1. The methods of determining the efficacy of an anti-cancer drug can further include the step of measuring the expression of Ki-67 in the multicellular tumoroid after exposure to the amount of the anti-cancer drug. The methods of determining the efficacy of an anti-cancer drug can also include the step of measuring the amount of VEGF or IL-6 present in the culture media after exposure to the amount of the anti-cancer drug.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A-1B show representative fluorescent microscopic images demonstrating growth of BT474 breast cancer cell derived tumoroids after about 5 days of culturing with (FIG. 1B) or without (FIG. 1A) cancer associated fibroblasts (CAFs) and endothelial cells (ECs).

FIG. 2 shows a confocal microscope image (merged z-stacked images) of BT474 breast cancer co-cultured tumoroids showing EVs (vWF, green cells) and CAFs (SMA positive, red cells) and nuclei (DAPI, blue).

FIG. 3 shows a graph demonstrating the amount of VEGF in the supernatant of the cultured cells of FIGS. 1A-1B and 2 as measured by ELISA. Cells were cultured for 5 days prior to determining the amount of VEGF produced by the cultured cells.

FIGS. 4A and 4B show representative fluorescent microscopic images demonstrating growth of HCC1569 breast cancer cell derived tumorids after about 5 days of culturing with (FIG. 4B) or without (FIG. 4A) cancer associated fibroblasts (CAFs) and endothelial cells (ECs). The results shown are from one representative experiment out of three.

FIGS. 5A-5C show graphs demonstrating VEGF (FIG. 5A), IL-6 (FIG. 5B), and active TGF-β1 (FIG. 5C) present in human mesenchymal stem cell (hMSC) conditioned medium (CM). Human MSCs were cultured in alpha-MEM and 15% serum. Conditioned medium of passages (p) p2-p6 was collected about 48 h after each passage. Collected conditioned medium was centrifuged, filtered (using a 0.45 μm filter), and stored at −80° until use. Expression of the growth factors in the CM of p2-p6 was examined by ELISA. Neutralizing VEGF antibody was used to demonstrate specificity of VEGF. * p<0.05.

FIGS. 6A-6H demonstrate representative fluorescent microscope images of monoculture (FIGS. 6A-6D) of BT474 breast cancer cells or co-culture (FIGS. 6E-6H) of BT474 breast cancer cells and ECs+CAFs stained by Calcein-AM/EthD-1 to detect live (green) and dead (red) cells. Magnification: 100×. The ratio of standard growth medium (GM) to CM (GM:CM) ranged from about 100:0 to about 50:50.

FIG. 7 demonstrates tumoroid diameter of the tumoroids shown in FIGS. 6A-6H. Tumoroid diameter was measured using Image J. Three scaffolds/group and 10 tumorids/scaffold examined * p<0.05.

FIGS. 8A to 8B demonstrate BT474 tumoroid cultures stained with calcein-AM/EthD-1 that stains live cells green and dead cells red (FIG. 8A) and Ki-67 staining for a monoculture of BT474 on a 3-D scaffold (FIG. 8B).

FIGS. 9A and 19B show graphs demonstrating differential responses to Lapatinib in BT474 (FIG. 9A) and HCC1569 (FIG. 9B) tumoroids grown in monoculture or co-culture. On day 2 after culturing, cells were treated with the indicated concentrations (μM) of Lapatinib for about 72 hours. Cell viability was measured by PrestoBlue® assay.

FIGS. 10A and 10B show representative images that demonstrate Ki-67 expression in Lapatinib treated BT474 (FIG. 10A) co-cultured tumoroids or control untreated tumoroids (FIG. 10B). On day 2 after co-culture, cells were treated with 2.5 μM of Lapatinib for 72 hours, and Ki-67 expression in the control and Lapatinib treated culture was determined by immunohistochemistry.

FIGS. 11A-11C show graphs demonstrating the effect of Lapatinib on VEGF (FIG. 11A), IL-6 (FIG. 11B), and TGF-β1 (FIG. 11C) on BT474 tumoroids derived from monocultures or co-culturing BT474 cells with CAFs and ECs on a 3-D scaffold. BT474 tumoroids were monocultured or co-cultured in the presence or absence of Lapatinib (2.5-10 μM) and the levels of VEGF, IL-6, and TGF-β1 in the day 5 culture supernatants were determined by ELISA. * p<0.05.

FIGS. 12A-12D demonstrate breast cancer cells forming SCTs when cultured on the FiSS. Cells (10×10³) were cultured for about 5 days on FiSS. Tumoroids formed were stained with calcein AM/EthD-1 that stains live cells green and dead cells red.

FIG. 13 shows a graph demonstrating the growth of breast cancer cells on the FiSS. Cells (10×10³) were cultured in triplicates on the scaffold for 9 days on the FiSS. Tumoroid size was measured by ImageJ analysis.

FIGS. 14A-14D demonstrate the growth of breast cancer cells co-cultured with CAFs and/or ECs on the FiSS. Day-5 MCF-7 or HCC-1569 tumoroids were cultured with ECs or CAFs (5×10³) or a combination of ECs and CAFs (2.5×10³ each) in triplicates on the FiSS for another 4 days. MCTs at day 9 were stained with calcein AM/EthD-1 that stains live cells green and dead cells red.

FIG. 15 shows a graph demonstrating the results of a Presto Blue assay to evaluate the growth of breast cancer cells co-cultured with CAFs and/or ECs on the FiSS. Growth of MCTs was monitored using Presto Blue Assay at day-9.

FIGS. 16A-16F demonstrate calcein stained SCTs (FIGS. 16A-16C) and MCTs (FIGS. 16D-16F) derived from MCF7 (FIGS. 16A and 16D), HCC1569 (FIGS. 16B and 16F), and BT474 (FIGS. 16C and 16F) breast cancer cell lines. Tumor cells were mono-cultured (upper) or co-cultured with CAFs and ECs on FiSS and stained with calcein AM/EthD-1 that stains live cells green and dead cells red. Day-5 SCTs and MCTs derived from MCF7, HCC1569 and BT474 are shown.

FIGS. 17A-17F demonstrate representative images of fixed tumoroids that were immunostained for VWF and SMA and counterstained by DAPI. Representative fluorescent images (FIGS. 17A-17C) and confocal microscopic images (merged z-stacked images) (FIGS. 17D-17F) of MCTs showing ECs (vWF, green cells), CAFs (SMA positive, red cells) and DAPI (total cell nuclei) are shown.

FIGS. 18A-18D demonstrate representative images of a comparison of FiSS induced SCTs with colonies formed by Matrigel based 3D culture. Cells (3×10³) were cultured on a layer of Matrigel coated plate in the presence of growth medium supplemented with 10% Matrigel. Cells were cultured for 5 days and spheroids were stained with calcein AM and examined by fluorescent microscope. FIGS. 18A and 18C show colonies formed on the Matrigel and FIGS. 18B and 18D show SCTs formed on FiSS. FIGS. 19A-19B show results from MCF7 cells and FIGS. 18C-18D show results from BT474 cells.

FIG. 19 shows a graph demonstrating VEGF expression in MCF7-SCTs and -MCTs. MCF7 tumoroids were cultured for 5 days and the levels of VEGF in the day 5 culture supernatants were determined by ELISA.

FIG. 20 shows an image of a scan data showing signal intensity of Array 1 and Array 2. Panels 1-8 contained controls with various standards for a standard curve. Panels 9-12 contained SCT samples. Panels 13-16 contained MCT samples.

FIG. 21 shows a Table with a list of antibodies used in the array of FIG. 20.

FIGS. 22A-22C show graphs demonstrating the protein expression of IL-6 (FIG. 22A), IL-8 (FIG. 22B), and MCP-1 (FIG. 22C) produced by SCT and MCT tumoroids. BT474 tumoroids (SCTs, blue bars and MCTs, red bars) were cultured in the presence or absence of Lapatinib (0.5-12.5 μM), as in FIGS. 9A-9B, and the levels of IL-6, IL-8, MCP-1 in the day 5 culture supernatants were determined by Sandwich ELISA (FIGS. 20-21). * p<0.05.

FIGS. 25A-25C show graphs demonstrating the protein expression of PDGF-BB (FIG. 25A), DKK-1 (FIG. 25B), and OPG (FIG. 25C) produced by SCT and MCT tumoroids. BT474 tumoroids (SCTs, blue bars and MCTs, red bars) were cultured in the presence or absence of Lapatinib (0.5-12.5 μM), as in FIGS. 9A-9B, and the levels of PDGF-BB, DKK-1, OPG in the day 5 culture supernatants were determined by Sandwich ELISA (FIGS. 20-21). * p<0.05.

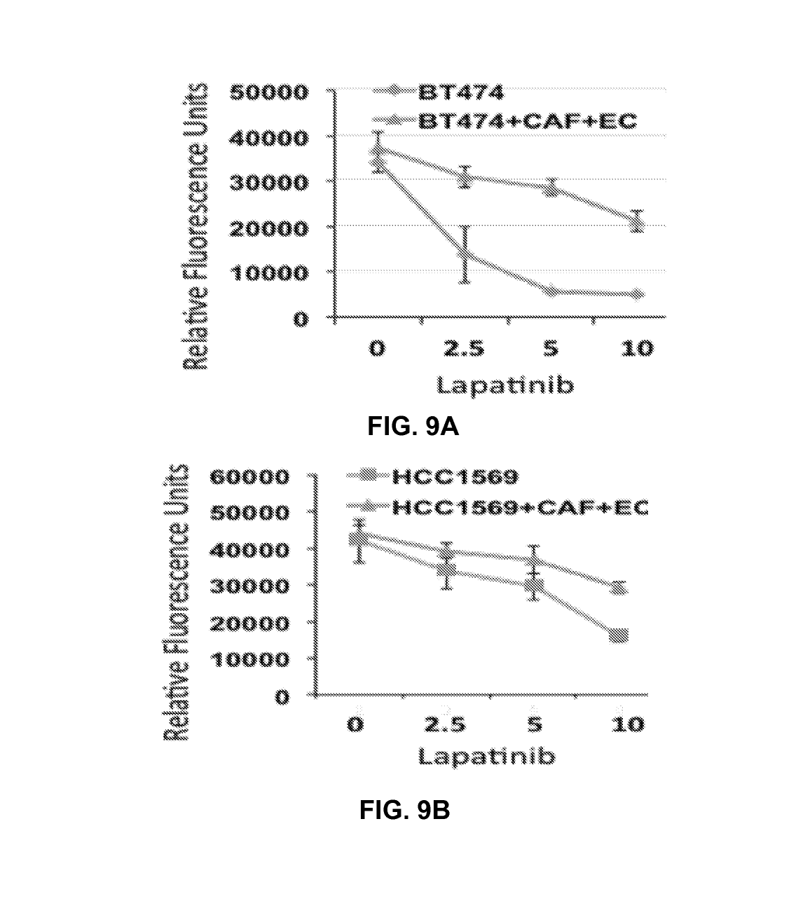

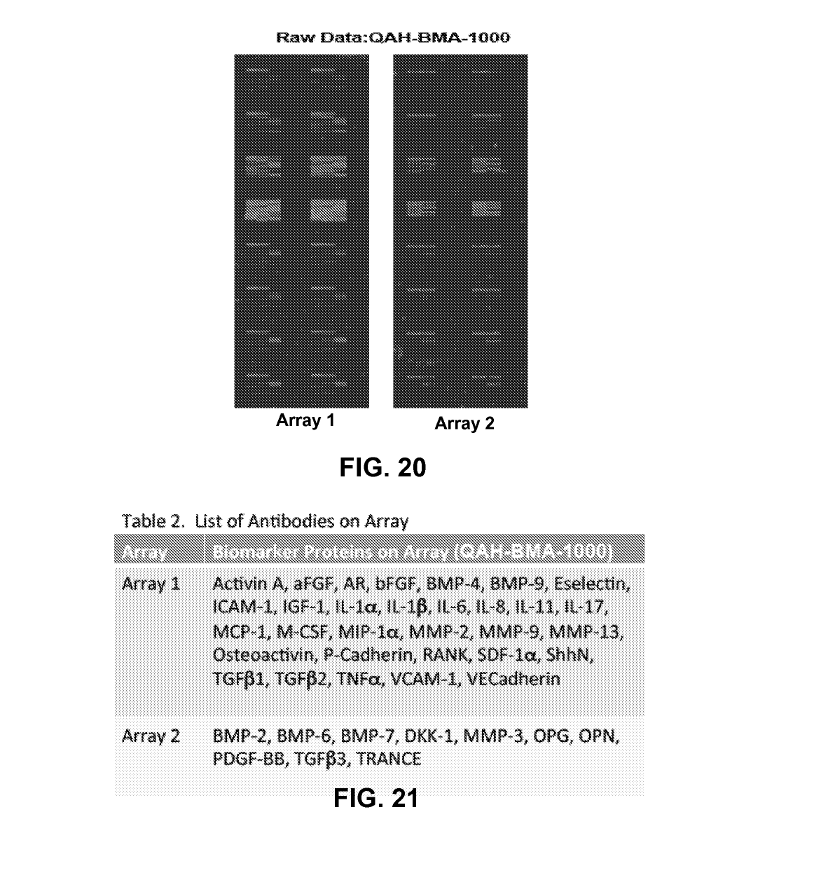

FIG. 24 shows a graph demonstrating the protein expression of MMP-3 produced by SCT and MCT tumoroids. BT474 tumoroids (SCTs, blue bars and MCTs, red bars) were cultured in the presence or absence of Lapatinib (0.5-12.5 μM), as in FIGS. 9A-9B, and the levels of MMP-3 in the day 5 culture supernatants were determined by Sandwich ELISA (FIGS. 20-21). * p<0.05.

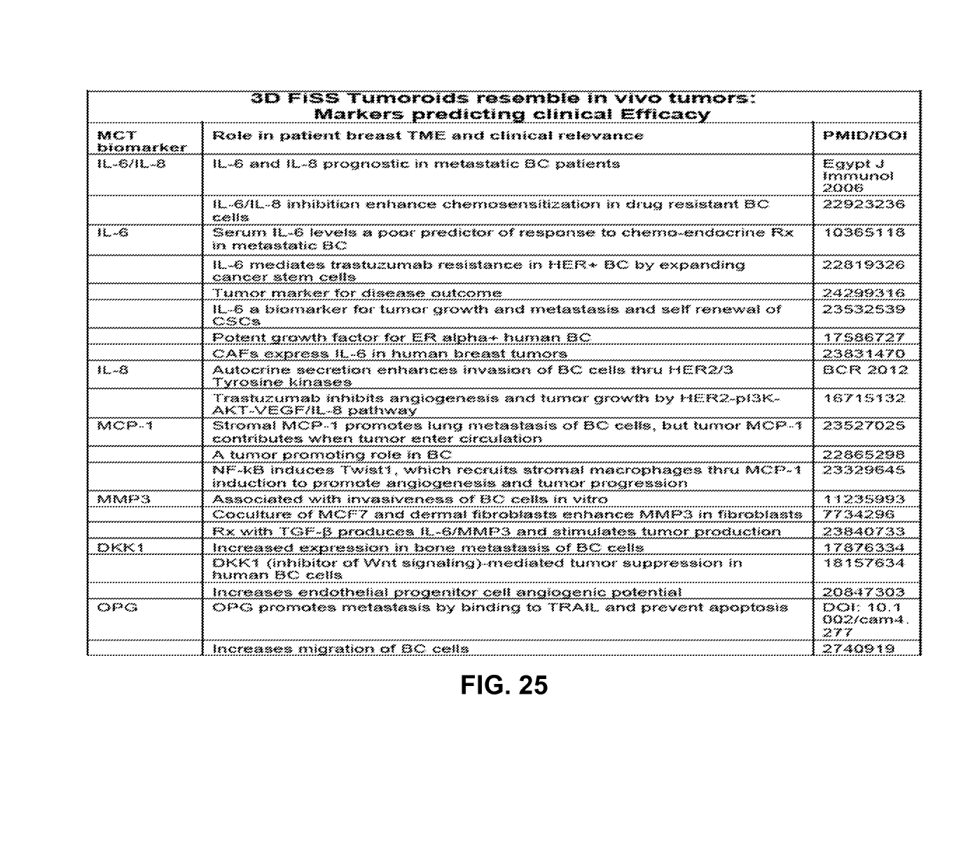

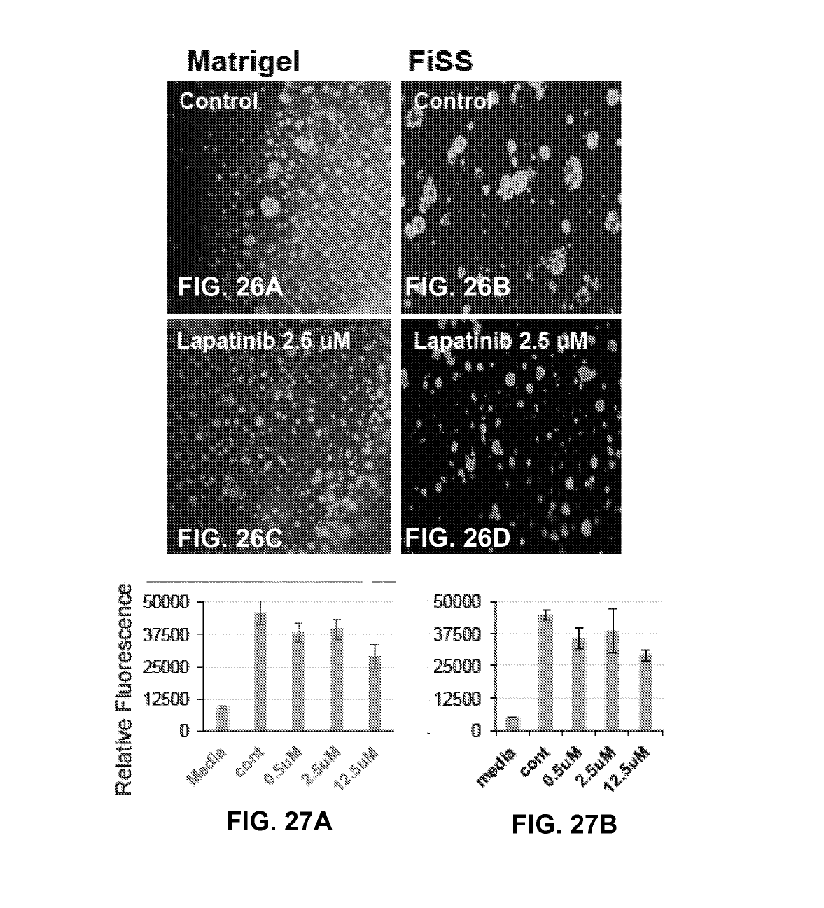

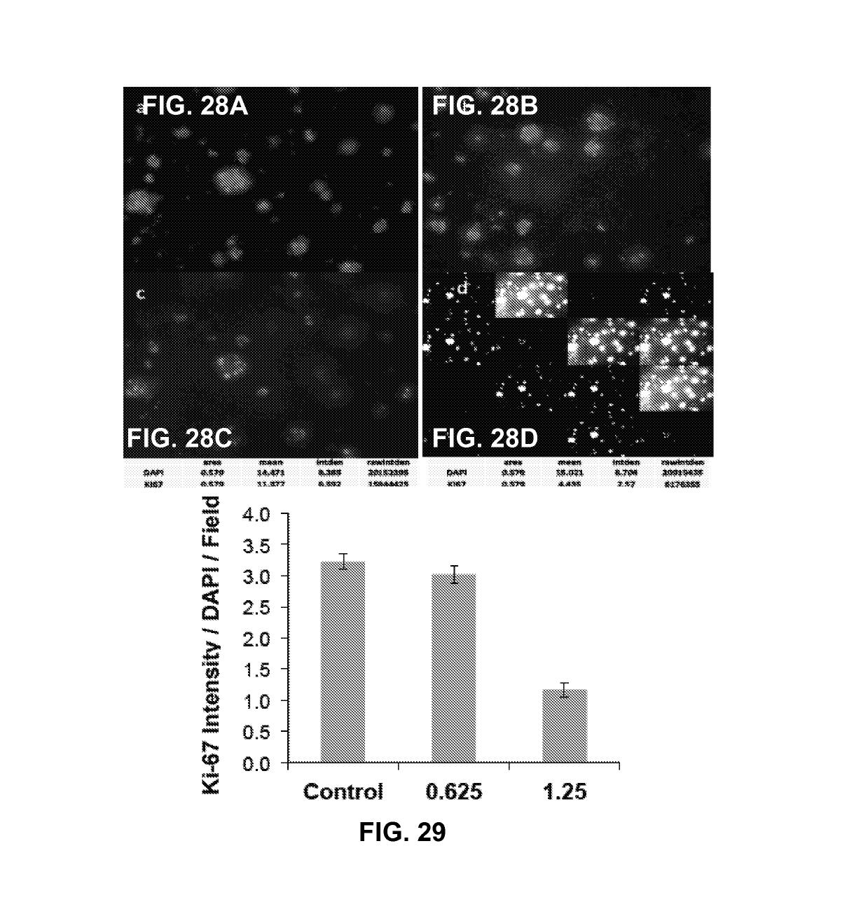

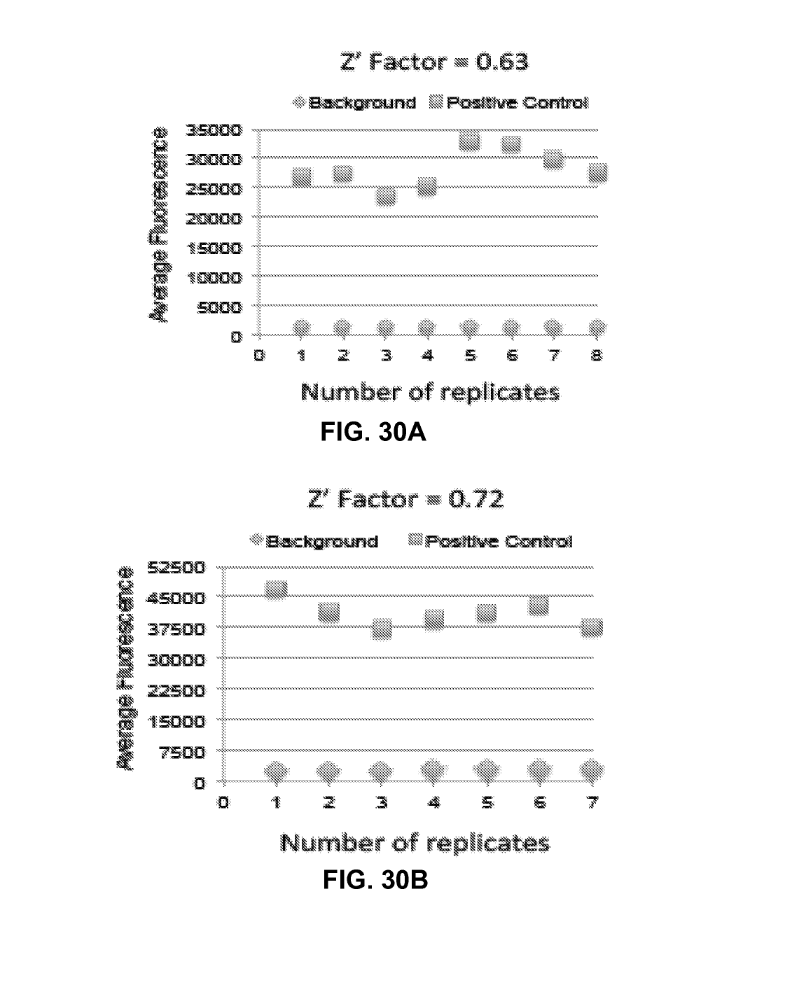

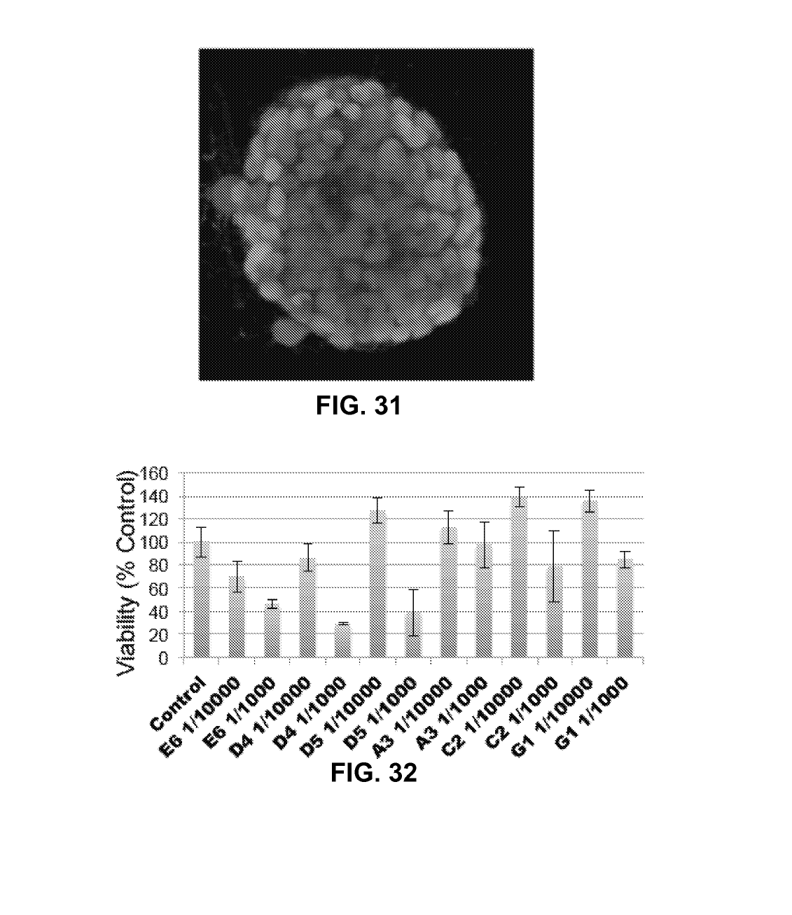

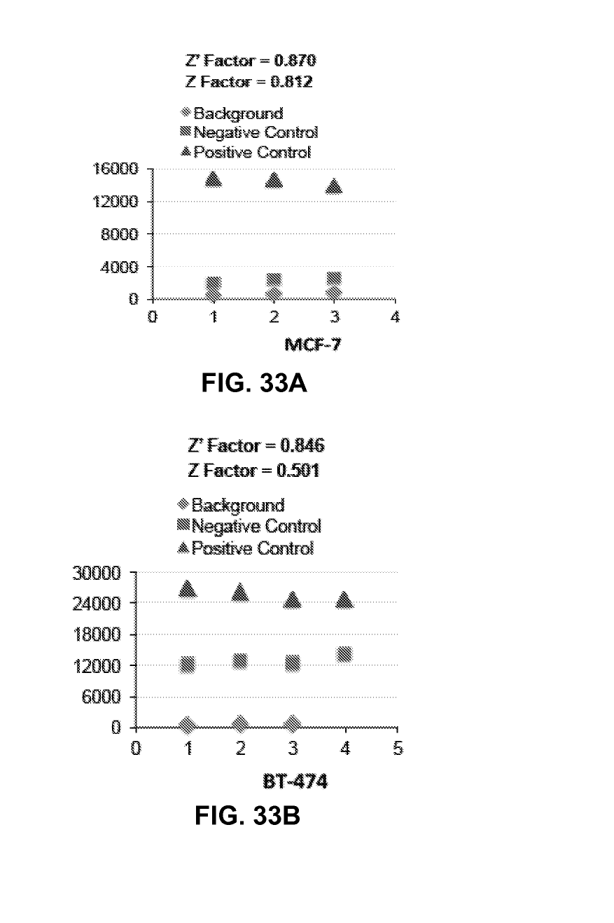

FIG. 25 describes various biomarkers that can be used to determine or predict clinical efficacy of a compound.

FIGS. 26A-26D show representative images of calcein AM stained tumoroids demonstrating the comparison of Lapatinib response to FiSS induced SCTs with Matrigel-based 3D culture. Cells (3×10³) were cultured on a layer of Matrigel coated plate in the presence of growth medium supplemented with 10% Matrigel. Three days after culture, cells were treated with indicated concentrations of Lapatinib and examined after 72 hrs. Spheroids were stained with calcein AM and examined by fluorescent microscope.

FIGS. 27A-27B show graphs demonstrating the results from a Presto Blue® assay to determine cell viability of the tumoroids cultured on Matrigel (FIG. 27A) or FiSS (FIG. 27B).

FIGS. 28A-28D show representative Z-stacked images demonstrating HCA using Operetta. The Z stacked images were acquired using Operetta (Perkin Elmer) and were subjected to image J (FIGS. 28A-28B) and image J threshold analyses (FIGS. 28C and 28D). The mean intensity for DAPI and Ki67 were determined.

FIG. 29 shows a graph demonstrating the results from culturing BT474 tumoroids in the presence or absence of Lapatinib for about 72 hrs, fixing the tumoroids, and immunostaining the tumoroids for Ki-67. Quantification of immunostaining was completed using Operetta. Relative Ki-67 intensity is demonstrated.

FIGS. 30A-30B demonstrate Z′-Factor analysis in tumoroid culture using PrestoBlue Assay. LLC1 cells were plated at 5000 cells/well in a 96 well plate pre-loaded with FiSS. Forty-eight hours later, wells were replenished with 100 μl of fresh media containing 10% DMSO (vehicle). After 72 hours, wells were incubated with PrestoBlue reagent and fluorescence was measured (BioTek Synergy plate reader).

FIG. 31 shows a confocal image of a representative SCT showing full Cell Titer-Glo reagent penetration.

FIG. 32 shows a graph demonstrating viability of BT474 cells (in replicates of 4 wells/group) determined using CellTiter-Glo 2.0 assay. Luminescence was measured using a BioTek Synergy plate reader. Data represent mean±SD.

FIGS. 33A-33B show graphs demonstrating the minimal well to well variation of MCF7 (FIG. 33A) and BT-474 (FIG. 33B) tumoroids cultured on fabricated FiSS-96 well microplates.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

### DEFINITIONS

As used herein, “tumoroid” refers to a micrometastatic compact aggregate of tumor cells. Tumoroids can respond to the same biochemical, nanotopographical, and mechanical cues that drive tumor progression in the extracellular matrix.

As used herein, the terms “cancer,” “cancer cells,” “neoplastic cells,” “neoplasia,” “tumor,” and “tumor cells” (used interchangeably) refer to cells which exhibit relatively autonomous growth so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures. The cancer can be selected from astrocytoma, adrenocortical carcinoma, appendix cancer, basal cell carcinoma, bile duct cancer, bladder cancer, bone cancer, brain cancer, brain stem glioma, breast cancer, cervical cancer, colon cancer, colorectal cancer, cutaneous T-cell lymphoma, ductal cancer, endometrial cancer, ependymoma, Ewing sarcoma, esophageal cancer, eye cancer, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer and Wilms tumor. In some embodiments, the cancer is prostate cancer.

The terms “cell,” “cell line,” and “cell culture” include progeny. It is also understood that all progeny may not be precisely identical in DNA content due to deliberate or inadvertent mutations. Variant progeny that have the same function or biological property, as screened for in the originally transformed cell, are included. The “host cells” used in the present invention generally are prokaryotic or eukaryotic hosts.

As used herein, “scaffold” refers to a three-dimensional porous sold biomaterial that can: (1) promote cell-biomaterial interactions, cell adhesion, and extracellular matrix deposition; (2) permit sufficient transport of gasses, nutrients, and/or regulatory factors to allow cell survival, proliferation, and/or differentiation; (3) biodegrade at a controllable rate that approximates the rate of tissue regeneration under culture conditions of interest; and/or (4) provoke a minimal degree of inflammation or toxicity if introduced in vivo.

As used herein, “stemness” refers to properties, characteristics (structural or functional), and molecular signatures that distinguish stem cells from other differentiated cell types.

As used herein, “stemness factors” can refer to genes or proteins that are required for or involved in stem cell self-renewal or other property or characteristic that is unique to stem cells, including but not limited to OCT-4, SSEAs, CD133, ABCG2, Nestin, Sox2, Naong, CD44, EpCAM (ESA, TROP1), CD24 (HSA), CD90, CD200, and ALDH.

As used herein “fibrous scaffold” refers to a three dimensional structure formed by randomly oriented fibers. In some embodiments, electrospining methods are used to achieve the randomly oriented fiber construction.

A “subject,” “individual,” or “patient,” used interchangeably herein, refers to a vertebrate, preferably a mammal, more preferably a human. Mammals include, but are not limited to, murines, simians, humans, farm animals, sport animals, and pets.

As used herein, “composition” refers to a combination of active agent and another compound or composition, inert (for example, a detectable agent or label) or active, such as an adjuvant.

As used herein, “control” is an alternative subject or sample used in an experiment for comparison purpose and included to minimize or distinguish the effect of variables other than an independent variable.

As used herein, “positive control” refers to a “control” that is designed to produce the desired result, provided that all reagents are functioning properly and that the experiment is properly conducted.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “culturing” refers to maintaining cells under conditions in which they can proliferate and avoid senescence as a group of cells. “Culturing” can also include conditions in which the cells also or alternatively differentiate.

As used herein, “expansion” or “expanded” in the context of cell refers to an increase in the number of a characteristic cell type, or cell types, from an initial population of cells, which may or may not be identical. The initial cells used for expansion need not be the same as the cells generated from expansion. For instance, the expanded cells may be produced by ex vivo or in vitro growth and differentiation of the initial population of cells.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, “concentrated” refers to a molecule, including but not limited to a polynucleotide, peptide, polypeptide, protein, antibody, or fragments thereof, that is distinguishable from its naturally occurring counterpart in that the concentration or number of molecules per volume is greater than that of its naturally occurring counterpart.

As used herein, “diluted” refers to a molecule, including but not limited to a polynucleotide, peptide, polypeptide, protein, antibody, or fragments thereof, that is distinguishable from its naturally occurring counterpart in that the concentration or number of molecules per volume is less than that of its naturally occurring counterpart.

As used herein, “mesenchymal stem cell” or “MSC” refers herein to a multipotent cell capable of differentiating into cells that compose adipose, bone, cartilage, and muscle tissue.

As used herein, “biocompatible” or “biocompatibility” refers to the ability of a material to be used by a patient without eliciting an adverse or otherwise inappropriate host response in the patient to the material or a derivative thereof, such as a metabolite, as compared to the host response in a normal or control patient.

As used herein, “biodegradable” refers to the ability of a material or compound to be decomposed by bacteria or other living organisms or organic processes.

As used herein, “stem cell” refers to any self-renewing totipotent, pluripotent cell or multipotent cell or progenitor cell or precursor cell that is capable of differentiating into multiple cell types.

Discussion

Only an estimated 9% of drugs that enter clinical development receive market approval. Some of the reasons for the high failure rate of new drugs are a poor understanding of the biology behind human disease that leads to a lack of clinical efficacy, drug toxicity, and side effects. Potential anti-cancer drugs entering clinical development have about a 95% attrition rate despite the rising cost of new drug development (about 800 million). The high attrition rate can be attributed to approaches used for cancer drug discovery, testing cancer cell response and drug development using two-dimensional (2D) cell culture assays and in vivo animal models.

2D cell culture systems have several disadvantages with regard to tumor biology, including: (1) cells in the system exhibit unnatural morphology; (2) cells in the system have a lower viability and poor differentiation capability; (3) cells in the system have changes in gene and protein expression profiles that differ substantially from the profiles of the same genes and proteins when expressed in vivo; (4) cells in the system exhibit an artificial metabolism; and (5) fail to accurately predict how well a new drug will perform in a clinical trial.

Three dimensional cell culture systems and tumor models can overcome many of the deficiencies of 2D systems. Generally, 3D systems can have improved mimicry of in vivo tumor microenvironments, including physiology, structure, concentration gradients of signaling molecules, and composition, structure, and mechanical forces of the extracellular matrix over 2D in vitro cell culture systems ad models. Although 3D in vitro tumor model systems have been described, current 3D in vitro tumor model systems are not without limitations.

The multicellular tumor spheroid (MTS) model is currently considered the best validated 3D model system. The MTS model has been implemented using a variety of biological, natural, and synthetic substrates in the form of hydrogels, films, or scaffolds. Further, a number of different techniques, including liquid-overlay, spinner flask, gyratory rotation, and the hanging drop methods have been used to grow spheroids. Despite advancements, MTS models have yet to be widely adopted. Indeed, the median level of adoption of 3D cell culture was less than about 25% of all cell culture work. Industry demands models having biological relevance, wide applicability/versatility, high throughput, and scalability/automation at a low cost. Current 3D models, including current MTS models, do not meet these demands insofar as they require a long cultivation time, form spheroids with a wide size distribution, and difficult mechanical accessibility. Little is understood about the biological relevance of current MTS models in relation to their ability to mimic solid tumors or their interactions. Further, MTS models that use scaffolds that are of animal or human origin to overcome issues with biological relevance suffer from a risk of disease transmission and poor reproducibility, which severely limits their potential for use in determining clinical efficacy of candidate drug compounds.

Much effort has been expended in developing biomimetic scaffolds for 3D culture. The best-developed scaffolds currently are those constructed from polystyrene or PCL, which allow cells to grow on an artificial albeit biocompatible surface. One major limitation of these scaffolds is that a longer exposure to trypsin is required to pull the spheroids out of the scaffold during culture, which stresses the cells and make the results obtained from any experiment or efficacy test difficult at best to interpret. Fully synthetic scaffolds such as the RGD-modified PEG hydrogels can create artificial cell-cell or cell-matrix interactions rendering screening of drugs targeting tumor-stroma interactions difficult.

Non-scaffold based approaches, such as the hanging drop and magnetic nano-3D technologies, exist but are not without limitation. The hanging drop method limits the growth of spheroids to 500 μm in diameter because the cells in the center starve, become unstable, and die. Moreover, a hanging drop spheroid emerges from a single tumor cell and thus its biological relevance is questionable. The magnetic nano-3D system is very expensive and the spheroids generated this way also suffer from the same central necrosis as those produced via the hanging drop method. In both cases, the tumor microenvironment observed can be very different from the in vivo tumor microenvironment, where heterogeneity exists in the tumor cells and stromal cells.

With the deficiencies of current models in mind said, described herein are compositions, methods, and systems for 3D culture of a tumoroid model system that can facilitate drug efficacy testing in clinical and drug development settings. The 3D culture methods and systems described herein can Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Methods of Forming Multi-Cellular Tumoroids

Described herein are methods of forming multi-cellular tumoroids. The cell population of the tumoroids formed can be heterogeneous (i.e. include different cell types). The methods can result in large tumoroids (greater than about 500 μm in diameter) that mimic the in vivo tumor microenvironment. In some embodiments, the method can include co-culturing tumor cells (TCs), cancer associated fibroblast cells (CAFs), and epithelial cells (ECs) in a cell culture medium. In some embodiments, the co-culture also can include macrophages. In some embodiments, the cell co-culture only contains TCs, CAFs, and ECs. In other embodiments, the cell co-culture only contains TCs, CAFs, ECs, and macrophages. The cell culture can continue through one or more passages of cells until a tumoroid of a desired size is formed. The tumoroid formed can be about 1 to about 500 μm, greater than 500 μm, or 500 μM to about 1,000 μM in diameter. In any given culture, the tumoroid size can be substantially uniform.

The cell culture medium can also contain an amount of a mesenchymal stem cell (MSC) conditioned media. In some embodiments, the co-culture of cells are cultured on a 3D scaffold. The 3D scaffold can be a fibrous induced smart scaffold (FiSS). As used herein the term FiSS refers to the 3D fibrous scaffold (also referred to as a 3P scaffold) described in Girard et al. (2013) PlosONE 8(10) e75345, which is incorporated herein by reference as if expressed in its entirety.

Co-Culture of Cells

TCs, CAFs, ECs, and Macrophages can be co-cultured as described herein. In some embodiments, the ratio of TCs to CAFs to ECs can be about 5 to about 1 to about 1. Stated differently, the ratio total number of each of the TCs, CAFs, and ECs in the culture can be present at a ratio of about 5 to about 1 to about 1. The ratio of TCs to CAFs to ECs to can range from about 1 to about 10:about 1 to about 10:about 1 to about 10. The ratio of TCs to CAFs to ECs to macrophages can range from 5:1:1:1. The ratio of TCs to CAFs to ECs to macrophages can range from about 1 to about 10:about 1 to about 10:about 1 to about 10:about 1 to about 10. The TCs, CAFs, ECs, and macrophages can be autologous, heterologous, or combinations thereof.

TCs:

The co-culture can include tumor cells. In some embodiments the tumor cells can include breast cancer cells. In other embodiments, the tumor cells can be only breast cancer cells. In some embodiments, the tumor cells can include lung cancer cells. In other embodiments, the tumor cells are only breast cancer cells. The TCs can be derived from a subject, such as via a biopsy of a tumor. In some embodiments, the biopsy is used directly as the source of TCs (i.e. by culturing the biopsy directly in the co-culture). In other embodiments, the biopsy can be cultured in vitro and TC progeny cells from the in vitro biopsy culture can be used as the source for the TCs. TCs can be standard cell lines used for clinical efficacy studies or other cell line that is commercially available. The TCs can be present at a ratio of TCs to macrophages of 1:1 to 10 to 1 to 1 to 10. The TCs can be present at a ratio of TCs to ECs of 1:1 to 10 to 1 to 1 to 10. The TCs can be present at a ratio of TCs to CAFs of 1:1 to 10 to 1 to 1 to 10.

CAFs:

The co-culture can include CAFs. In vivo, CAFs actively participate in the growth and invasion of tumor cells by providing a unique tumor microenvironment. CAFs can stem from trans-differentiation of resting resident fibroblasts or pericytes within the tumor microenvironment via mesenchymal transition. CAFs can be derived from bone marrow mesenchymal stem cells, from normal or transformed epithelial cells via epithelial to mesenchymal transition, and/or from endothelial cells via endothelial to mesenchymal muscle actin (SMA).

Tumor progression needs a positive and reciprocal feedback between CAFs and tumor cells. Cancer cells can induce and maintain the fibroblasts active phenotype, which in turn, produce a series of growth factors and cytokines that sustain tumor progression by promoting extracellular matrix (ECM) remodeling, cell proliferation, angiogenesis, maintenance of stemness, regulating inflammation, regulating immune response, promoting a hospitable metabolic environment and epithelial-mesenchymal transition. Such growth factors can include hepatocyte growth factor (HGF), transforming growth factor β (TGF-β), epidermal growth factor (EGF), stromal derived factor 1 (SDF-1), basic fibroblast growth factor (b-FGF), and vascular endothelial growth factor (VEGF). Indirectly, CAFs can promote and maintain tumor progression by secreting proteases and other molecules involved in proteolysis and degradation of the ECM, such as plasminogen activators and matrix metalloproteinases. This can result in the release of growth factors and cytokines previously discussed that can sustain tumor progression. CAFs can also have pleiotropic functions on immune cells. As previously discussed the variety of growth factors, cytokines, and chemokines secreted by CAFs can cause a strong inflammatory yet immunosuppressive environment.

The CAFs can be derived from a subject, such as via a biopsy of a tumor. In some embodiments, the biopsy is used directly as the source of CAFs. In other embodiments, the biopsy can be cultured in vitro and progeny CAFs from the in vitro biopsy culture can be used as the source for the CAFs. CAFs can be standard cell lines used for clinical efficacy studies or other CAF cell line that is commercially available. The CAFs can be present at a ratio of CAFs to macrophages of 1:1 to 10 to 1 to 1 to 10. The CAFs can be present at a ratio of CAFs to ECs of 1:1 to 10 to 1 to 1 to 10. The CAFs can be present at a ratio of CAFs to TCs of 1:1 to 10 to 1 to 1 to 10.

ECs:

The co-culture can contain ECs. The ECs can be present at a ratio of ECs to macrophages of 1:1 to 10 to 1 to 1 to 10. The ECs can be present at a ratio of ECs to CAFs of 1:1 to 10 to 1 to 1 to 10. The ECs can be present at a ratio of ECs to TCs of 1:1 to 10 to 1 to 1 to 10.

Macrophages:

In some embodiments, the co-culture can also contain macrophages. The macrophages can be present at a ratio of TCs to macrophages of 1:1 to 10 to 1 to 1 to 10. The macrophages can be present at a ratio of ECs to macrophages of 1:1 to 10 to 1 to 1 to 10. The macrophages can be present at a ratio of CAFs to macrophages of 1:1 to 10 to 1 to 1 to 10. Macrophages can promote tumorogenesis through inter alia promoting angiogenesis. CAFs can regulate immune cell recruitment and function. CAFs have been demonstrated to induce macrophage recruitment the tumor microenvironment and induce an immunosuppressive phenotype in macrophages via SDF-1 through stimulated by expression and/or secretion of MCP/CCL2, IL1-βILL-6, CXCL1, CXCL2, CXCL5, and CCL3.

3D-Culture Scaffold:

The aforementioned cells can be co-cultured on a 3D scaffold for an amount of time. The 3D scaffold can be fibrous induced smart scaffold (FiSS). As used herein the term FiSS refers to the 3D fibrous scaffold (also referred to as a 3P scaffold) described in Girard et al. (2013) PlosONE 8(10) e75345, which is incorporated herein by reference as if expressed in its entirety.

The scaffold can be fabricated by any suitable technique or method. Such techniques and methods include, without limitation, electro spinning, solvent casting/salt leaching, ice particle leaching, gas foaming/salt leaching, solvent evaporation, freeze drying, thermally induced phase separation, micromolding, photolithography, microfluidics, emulsification, decellularization processes, self-assemblies, microfiber wet spinning, melt-blown processing, sponge replication methods, simple calcium phosphate coating methods, inkjet printing, melt-based rapid prototyping processing or a combination thereof. One of skill in the art will appreciate that the technique(s) or method(s) used for scaffold fabrication will vary depending on, inter alia, the components present in the scaffold.

Scaffold materials can be synthetic, biologic, or combinations thereof. The scaffold materials can be degradable or nondegradable. The scaffold materials can be biocompatible. Synthetic scaffold materials can include, without limitation, PLA, PLG, PLGA, and PHA, PLLA, PGA, PCL, PDLLA, PEE based on PEO, and PBT.

Cell Culture Media:

The co-culture of cells are cultured in a culture media. The culture medium can be altered over the time course of tumoroid formation. For example, the cell culture media can be replaced (such as when passing the cells) or supplemented during culturing. The replacement media can be the same formulation or have a different formulation that the prior media. Other media components can be supplemented to the media during culturing, which can result in a change in the media formulation.

The cell culture media can be a suitable standard base medium that can optionally be supplemented with, without limitation, growth factors, nutrients (e.g. nitrogen, glucose, amino acids), anti-fungals, antibiotics, ions, serum, and/or combinations thereof. Suitable base mediums include without limitation, DMEM, DME, RMPI-1640, and MEM. Others will be appreciated by those in the art.

In some embodiments, the culture media is supplemented with about 5 to about 10% Matrigel. The cell culture media can be supplemented with VEGF, IL6, TGF-β1, or combinations thereof. In some embodiments, the amount of VEGF can be at least 800 pg/mL, can range from about 1 to about 1200 pg/mL, about 100 pg/mL to about 1200 pg/mL, or about 800 pg/mL to about 1200 pg/mL. In some embodiments, the amount of IL6 can be at least 100 pg/mL, can range from about 1 to about 500 pg/mL, about 100 pg/mL to about 500 pg/mL, or about 200 pg/mL to about 500 pg/mL. In some embodiments, the amount of TGF-β1 can be at least 1200 pg/mL and can range from about 1 to about 1800 pg/mL, about 900 pg/mL to about 1800 pg/mL, or about 1200 pg/mL to about 1800 pg/mL.

In some embodiments, the cell culture media is made of a growth media configured to promote growth of the tumoroid and a conditioned media. Formulations for the growth media will be appreciated by those of skill in the art. The conditioned media can be present at a concentration of about 1% to about 99% of the total culture media. In some embodiments the conditioned media is at least 20% of the total culture media. In further embodiments, the conditioned media can be about 20% to about 50% of the total cell culture media.

The conditioned media can be a human mesenchymal stem cell (MSC) conditioned media. MSC conditioned media can be obtained by culturing human MSC cells for one or more passages and collecting the media that the MSC cells were cultured in. In some embodiments, the MSC condition media is obtained from cell culture media collected at passaged 5 and/or passage 6. The MSC conditioned media can contain molecules and other compounds secreted by the MSC cells. In some embodiments the MSC media can contain VEGF, IL6, TGF-β1, or combinations thereof. In some embodiments, the amount of VEGF in the MSC conditioned media can be at least 800 pg/mL, can range from about 1 to about 1200 pg/mL, about 100 pg/mL to about 1200 pg/mL, or about 800 pg/mL to about 1200 pg/mL. In some embodiments, the amount of IL6 in the MSC conditioned media can be at least 100 pg/mL, can range from about 1 to about 500 pg/mL, about 100 pg/mL to about 500 pg/mL, or about 200 pg/mL to about 500 pg/mL. In some embodiments, the amount of TGF-β1 in the MSC conditioned media can be at least 1200 pg/mL and can range from about 1 to about 1800 pg/mL, about 900 pg/mL to about 1800 pg/mL, or about 1200 pg/mL to about 1800 pg/mL.

Methods of Using Multi-Cellular Tumoroids

The tumoroids formed as described herein can be used to determine the efficacy and/or effect of a compound or composition, such as a drug, including anti-cancer drugs or pharmaceuticals. As such, the methods and tumoroids described herein can be useful as model systems for clinical trials and drug discovery. In embodiments where the tumoroid is formed from a subjects own tumor, the efficacy of a particular treatment regimen can be examined. In some embodiments, the tumoroids and culture methods described herein can be used to expand the in vitro population of cancer stem cells.

The methods of determining the efficacy and/or effect of a compound or composition, such as an anti-cancer drug, can include the step of forming a multicellular tumoroid, where the step of forming the tumoroid as described anywhere herein, and exposing the multicellular tumoroid to an amount of a compound or composition, such as an anti-cancer drug. The step of forming the tumoroid can include the step of co-culturing TCs, ECS, and CAFs in a cell culture media, where the ratio of the number of TCs to CAFs to ECs is 5 to 1 to 1, and where the cell culture media contains an amount of a mesenchymal stem cell conditioned media. The cell co-culture can also optionally contain macrophages. In some embodiments, the cell co-culture only contains TCs, CAFs, and ECs. In other embodiments, the cell co-culture only contains TCs, CAFs, ECs, and macrophages.

The method of determining the efficacy and/or effect of a compound or composition, such as an anti-cancer drug, can optionally include the step of measuring the amount of expression or presence of a biomarker indicative of tumor growth or stemness and comparing the expression or presence to that of a suitable control. A changed in the amount (either increased or decreased amount) of expression or presence of the biomarker in the sample tested can indicate efficacy or inefficacy of the compound or composition against the cancer. The biomarker can be measured in the tumoroid itself or in the culture media that the tumoroid is present in. In some embodiments, the method further includes the step of measuring the expression of Ki-67 in the tumoroid and comparing the expression to a suitable control. A decrease in the expression and/or presence of Ki-67 as compared to the control can indicate that the test compound or composition is effective against the cancer. In other embodiments, the method can further include the step of measuring the amount of VEGF and/or IL-6 present in the culture and comparing the expression or presence to a suitable control. A decrease in the expression and/or presence of VEGF and/or IL-6 as compared to the control can indicate that the test compound or composition is effective against the cancer.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

### Optimizing Tumoroid Assay (Z Factor Analysis)

A major stumbling block in 3D cell cultures has been reproducibility, which is important for its incorporation into high-throughput system screening. To determine well-to-well variation in the tumoroid assay, MCF-7 cells on FiSS in different concentrations in n=8 wells/group and measured the tumorigenicity five days after seeding by a PrestoBlue® assay. Z factor, a measure of statistical effect size, was determined using Equation 1.

$$Z \text{-} factor = 1 - \frac{3 ( {σ}_{p} + {σ}_{n} )}{\vert {μ}_{p} - {μ}_{n} \vert} . \qquad Equation 1$$

The assay results showed a small well-to-well variation with the Z factor being about 0.755, which was in the excellent range suggesting the readiness of the tumoroid assay described herein for high-throughput screening.

### Example 2

### Characterization of Tumoroid Co-Cultures

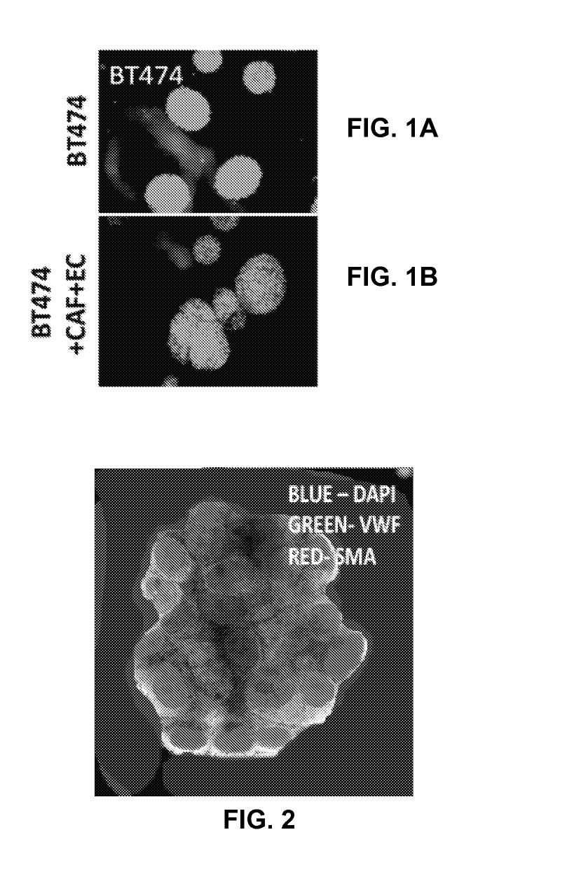

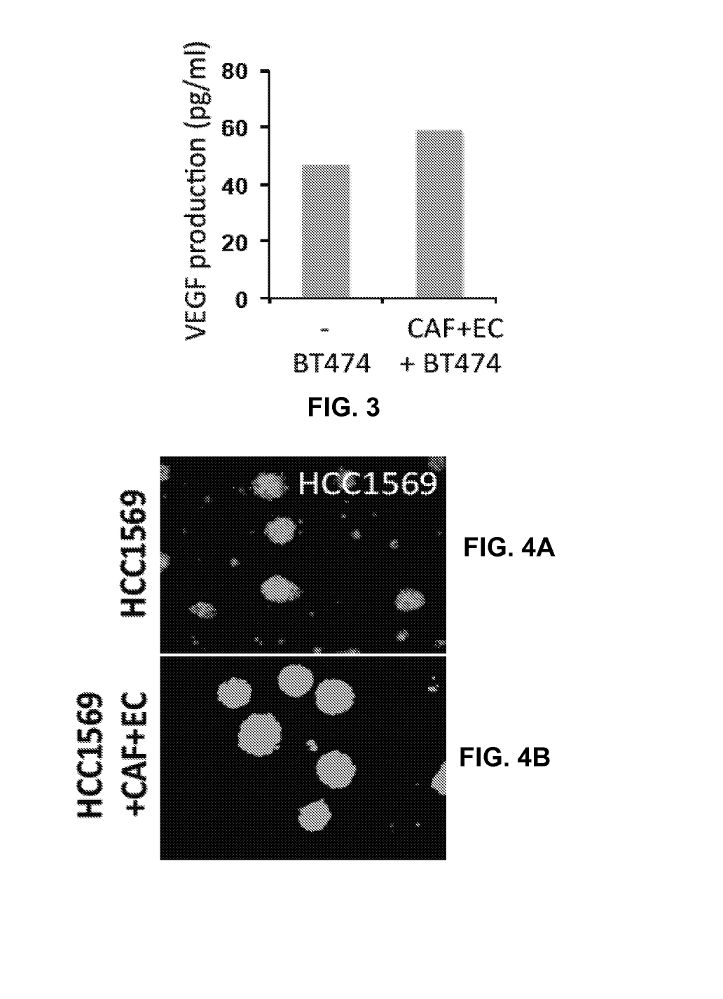

BT474 or HCC1569 cells were co-cultured with ECs and CAFs. The ratio of tumor cells (BT474 or HCC1569) to ECs to CAFs in the co-culture was 5:1:1 (tumorcells:ECs:CAFs). As demonstrated in FIGS. 2A-5B, co-culture with ECs and CAFs induced robust tumoroids that had increased growth potential and high VEGF expression (FIG. 3). FIGS. 1A-1B show representative fluorescent microscopic images demonstrating growth of BT474 breast cancer cell derived tumoroids after about 5 days of culturing with (FIG. 1B) or without (FIG. 1A) cancer associated fibroblasts (CAFs) and endothelial cells (ECs).

The presence of CAFs and ECs in the multi-cell tumoroids was confirmed by IHC using anti-smooth muscle actin (SMA) and anti-von Wille brand factor (vWF) antibodies for CAFs and ECs, respectively followed by confocal microscopy. FIG. 2 shows a confocal microscope image (merged z-stacked images) of BT474 breast cancer co-cultured tumoroids showing EVs (vWF, green cells) and CAFs (SMA positive, red cells) and nuclei (DAPI, blue). The merged z-stacked image of tumoroid immunostained form CAFs (red, anti-SMA positive) and ECs (green, anti-vWF-positive) is shown in FIG. 2. CAFs were found dispersed throughout the tumoroid whereas ECs were found mostly on the edge of the multi-cell tumoroid at day 5 after co-culture.

FIG. 3 shows a graph demonstrating the amount of VEGF in the supernatant of the cultured cells of FIGS. 1A-1B and 2 as measured by ELISA. Cells were cultured for 5 days prior to determining the amount of VEGF produced by the cultured cells. A comparison of VGEF levels in the supernatant of tumoroids showed that the BT474-tumoroids had less VGEF compared to BT474+CAF+EC induced tumoroids (FIG. 3). As demonstrated in FIGS. 4A-4B, the co-culture using HC15969 also produced a similar increase in number and diameter of tumoroids as seen with BT474.

### Example 3

### Delivery and Control of Growth Factors

To investigate growth factors that can enhance tumoroid growth in the co-cultures described herein, the effects of increasing serum concentrations on tumoroid growth during culturing. Results (not shown) suggest that increasing the serum concentration does not significantly affect tumoroid growth. Adding Matrigel (about 5% to about 10%) to the growth media increased tumoroid development of HCC1569 cells when cultured on FiSS. This suggests that supplementation of growth medium (GM) with about 5% to about 10% Matrigel can enhance tumoroid growth from other types of breast cancer cells.

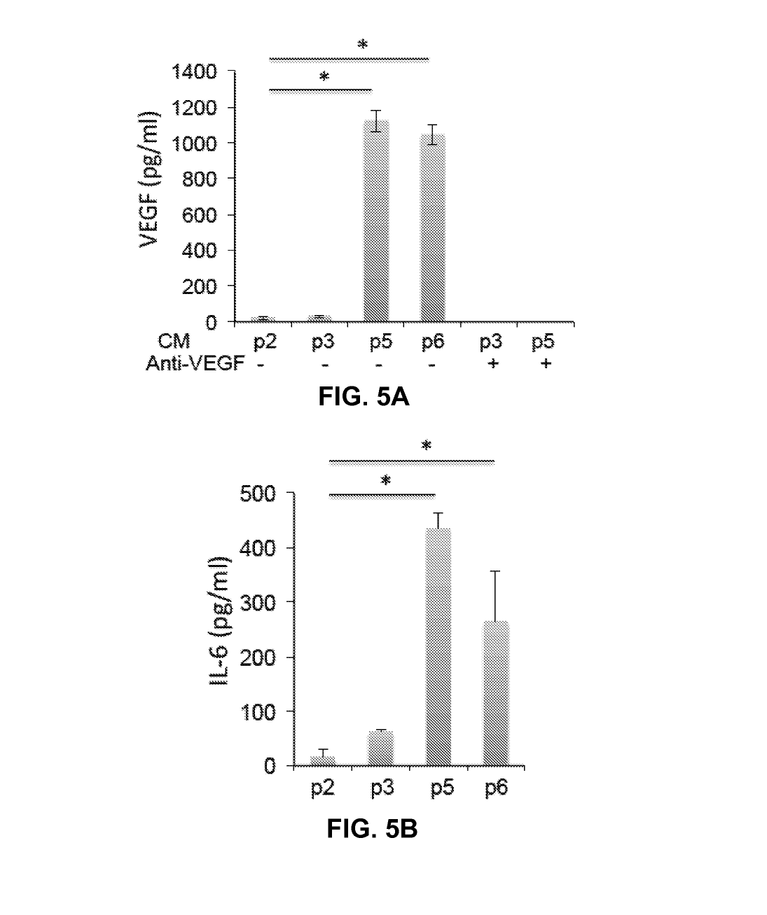

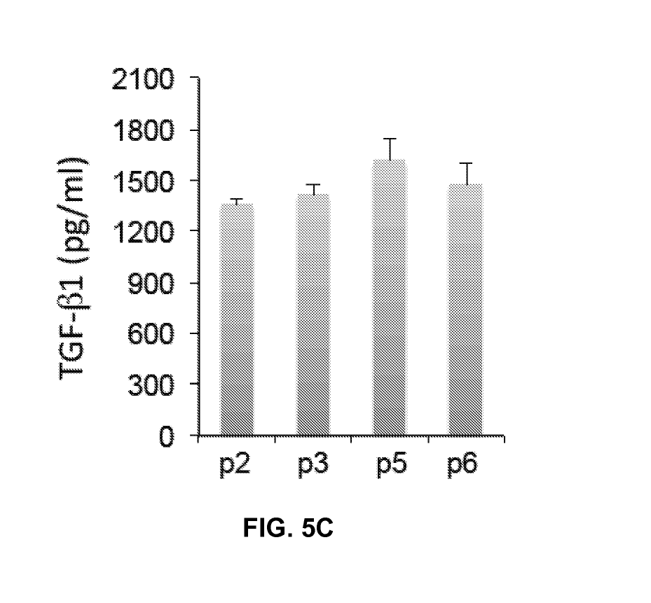

To test the factors that affect tumoroid growth, the effect of hMSC CM on tumoroid growth was examined. Therefore, growth factors released by hMSCs was examined. To this end, CM of hMSCs of different passages, such as passage 2, 3, 5 and 6, were collected and the levels of VEGF, IL-6, and TGF-8. FIGS. 5A-5C show graphs demonstrating VEGF (FIG. 5A), IL-6 (FIG. 5B), and active TGF-β1 (FIG. 5C) present in human mesenchymal stem cell (hMSC) conditioned medium (CM). Human MSCs were cultured in alpha-MEM and 15% serum. Conditioned medium of passages (p) p2-p6 was collected about 48 h after each passage. Collected conditioned medium was centrifuged, filtered, and stored until use. Expression of the growth factors in the CM of p2-p6 was examined by ELISA. Neutralizing VEGF antibody was used to demonstrate specificity of VEGF. * p<0.05. The data shown in FIGS. 5A-5C demonstrate significantly higher levels (about 1 ng/mL) of VEGF (FIG. 5A) and IL-6 (about 250-450 pg/mL) (FIG. 5B) were found in the CM of hMSCs-p5 and -p6 compared to -p2 and -p3. While present in CM, no significant difference in the levels of TGF-β1 was observed among CM of hMSC passages (FIG. 5C).

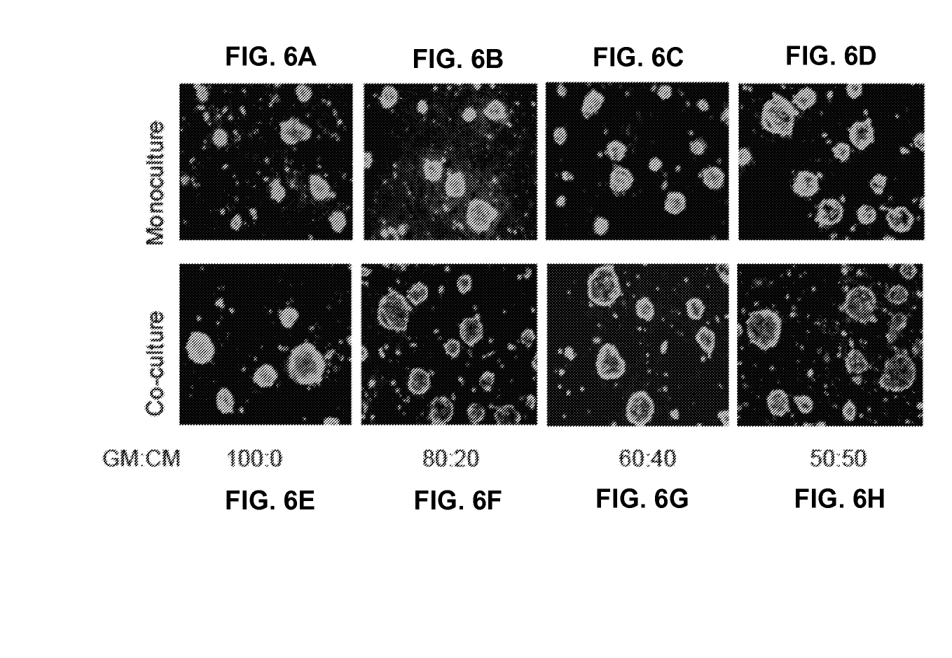

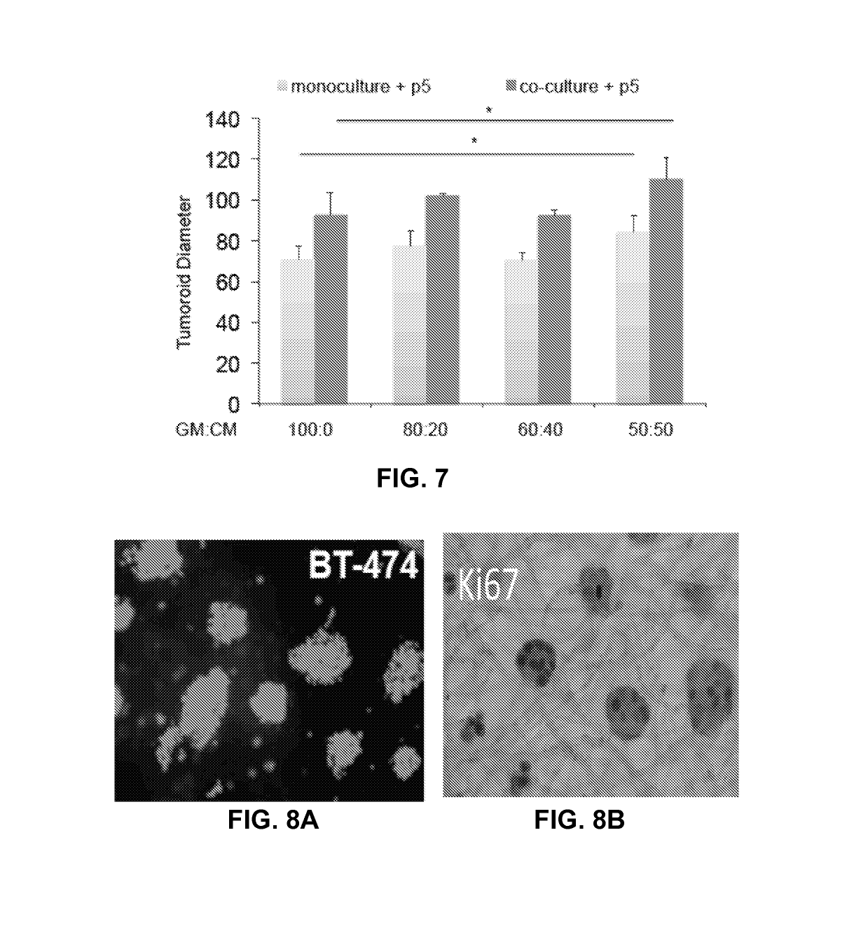

BT474 tumoroids were cultured from BT474 cells in the presence of varying concentrations of CM derived from hMSCs. Insofar as p5-hMSC CM showed greatest production of VEGF, IL-6, and TGF-β1, BT474 GM was supplemented with varying concentrations of p5-hMSC CM and cultured with BT474 cells only (monoculture) or in the presence of CAFs and ECs (co-culture) and tumoroid growth was examined. FIGS. 6A-6H demonstrate representative fluorescent microscope images of monoculture (FIGS. 6A-6D) of BT474 breast cancer cells or co-culture (FIGS. 6E-6H) of BT474 breast cancer cells and ECs+CAFs stained by Calcein-AM/EthD-1 to detect live (green) and dead (red) cells. Magnification: 100×. The ratio of standard growth medium (GM) to CM (GM:CM) ranged from about 100:0 to about 50:50. The data shown in FIGS. 6A-7, demonstrate that the addition of CM at about 20, about 40, or about 50% increased the tumoroid diameter in both monocultures and co-cultures. Addition of GM:CM at about 50% significantly increased tumoroid diameter for both tumoroids produced under monoculture culture and co-culture tumoroids.

### Example 4

### Biomarkers for Determination of Clinical Efficacy

Ki67 as a Clinical Biomarker of Tumoroids.

Clinical studies utilizes Ki67 as a marker for clinical efficacy. To determine whether Ki67 is expressed in tumoroids, BT474 cells (10⁴) were cultured for 5 days on FiSS. Tumoroids formed were fixed and immunostained using anti-Ki67 antibodies. FIGS. 8A to 8B demonstrate BT474 tumoroid cultures stained with calcein-AM/EthD-1 that stains live cells green and dead cells red (FIG. 8A) and Ki-67 staining for a monoculture of BT474 on a 3-D scaffold (FIG. 8B).

VEGF and IL6 as Biomarkers of Tumoroids.

The monoculture tumoroids and co-culture tumoroids produced significant amounts of VEGF and IL-6 released to the culture medium. The co-cultures had a 2-fold increase in the amount of VGEF and IL-6 released to the culture medium. The increase in the levels of VGEF and IL-6 produced in co-cultures suggest that these growth factors may be involved in coin the increase proliferation of cells and growth of tumoroids observed in co-cultures. Therefore, the data suggests that these two proteins can serve as biomarkers of clinical efficacy.

### Example 5

### Prediction of Clinical Efficacy with Tumoroids Using Herceptin and Lapatinib

The sensitivity of tumoroids derived from monoculture or co-culture of BT474 and HCC1569 to Lapatinib was examined. Lapatinib is a dual small molecule tyrosine kinase inhibitor targeting EGFR and HER2. As demonstrated in the data presented in FIGS. 9A-9B, tumoroids had varying responsiveness to Lapatinib treatment. BT474 cells were observed to be sensitive to Lapatinib when cultured both on 2D or FiSS (IC50<2.5 μM), but in the presence of ECs and CAFs, MT474-MCTs were observed to have significantly higher resistance to Laptinib (IC50>10 μM). HCC1569 also showed an increase in resistance to Lapatinib when cultured in the presence of ECs and CAFs. Similar results were obtained when established tumoroids were treated with Lapatinib.

### Example 6

### Biomarkers of Clinical Efficacy

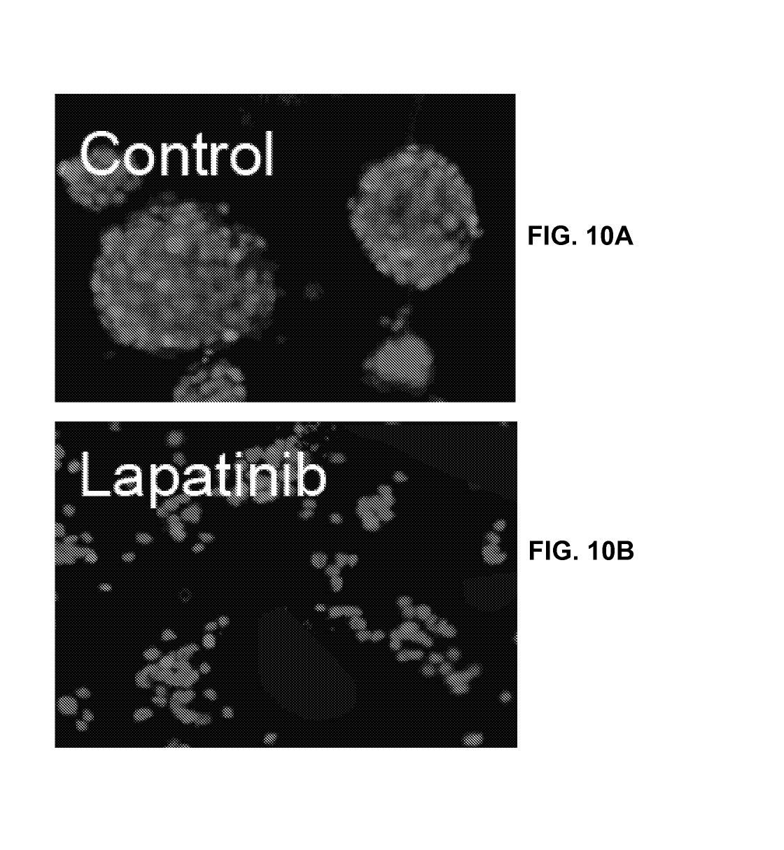

Ki-67 can be used as a surrogate marker of clinical efficacy in cancer trials. Expression of Ki-67. Monoculture and Co-culture tumoroids were treated with increasing concentrations of Lapatinib (0-10 μM). Tumoroids were fixed and immunostained for Ki-667. It was tested whether treatment with Lapatinib (2.5 mM) would affect expression of Ki-67. Results demonstrate that Lapatinib treatment not only inhibited tumoroid formation, but also completely abrogated Ki-67 staining, which suggested complete inhibition of the proliferation of tumor cells (FIGS. 10A and 10B).

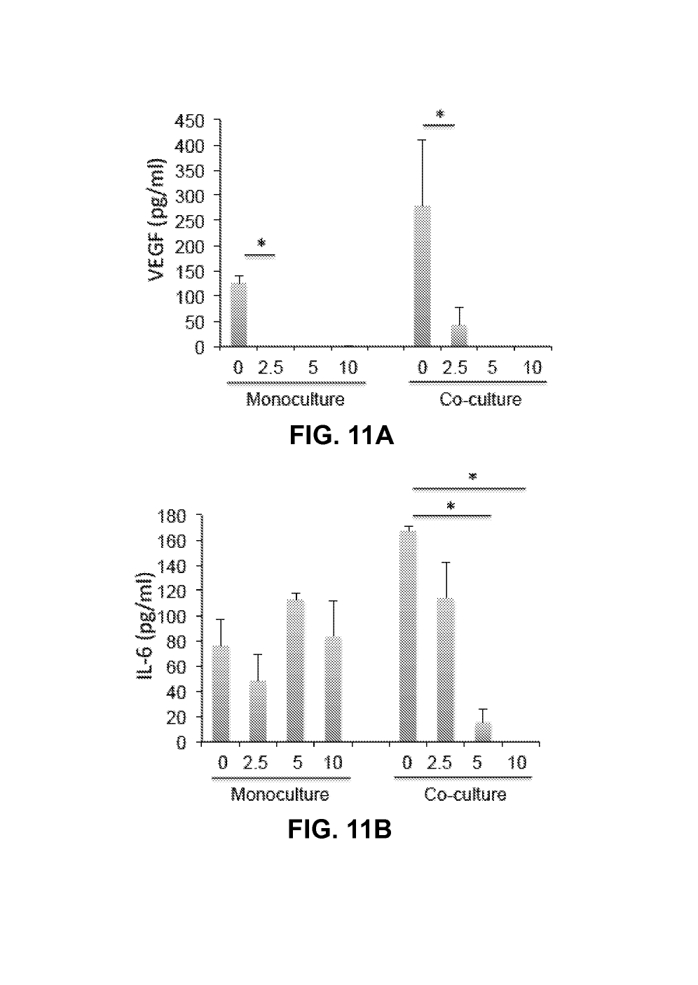

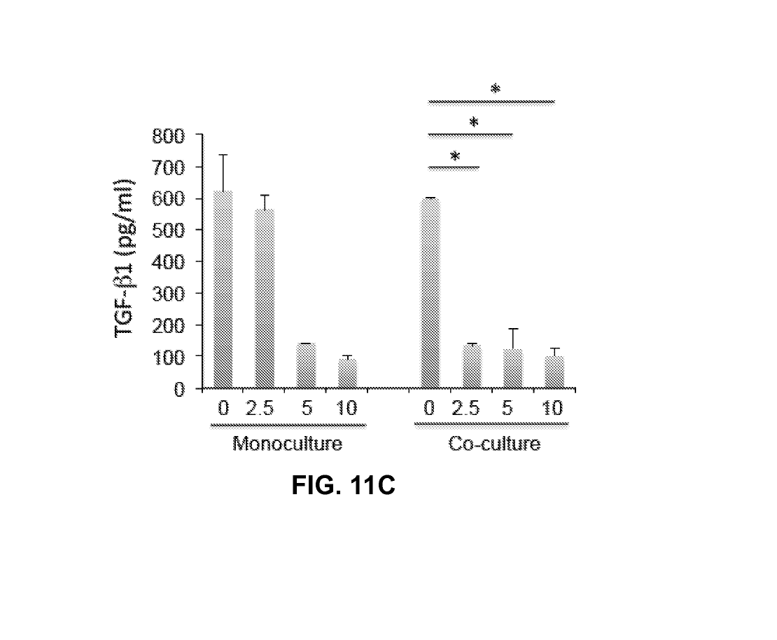

To identify and evaluate the biomarkers of clinical efficacy, both monoculture and co-culture tumoroids were treated with increasing concentrations of Lapatinib (about 0 to about 10 μM). Five days after culture, the supernatants of the culture were tested for levels of VEGF, IL-6, and TGF-β. FIGS. 11A-11C show graphs demonstrating the effect of Lapatinib on VEGF (FIG. 11A), IL-6 (FIG. 11B), and TGF-β1 (FIG. 11C) on BT474 tumoroids derived from monocultures or co-culturing BT474 cells with CAFs and ECs on a 3-D scaffold. BT474 tumoroids were mono-cultured or co-cultured in the presence or absence of Lapatinib (2.5-10 μM) and the levels of VEGF, IL-6, and TGF-β1 in the day 5 culture supernatants were determined by ELISA. * p<0.05. The data demonstrate that both monoculture and co-culture tumoroids secreted substantial amounts of VEGF, IL-6, and TGF-β. Lapatinib treatment (about 2.5 μM-5 μM) significantly decreased the secretion of VEGF, IL-6, and TGF-β in the co-cultured tumoroids. The data demonstrates that in addition to Ki67, these factors can serve as biomarkers of clinical efficacy of anti-cancer drugs in co-cultured tumoroids.

### Example 7

### Co-Culture of Breast Cancer Tumor Cells with Stromal Cells and Assessment of TSI

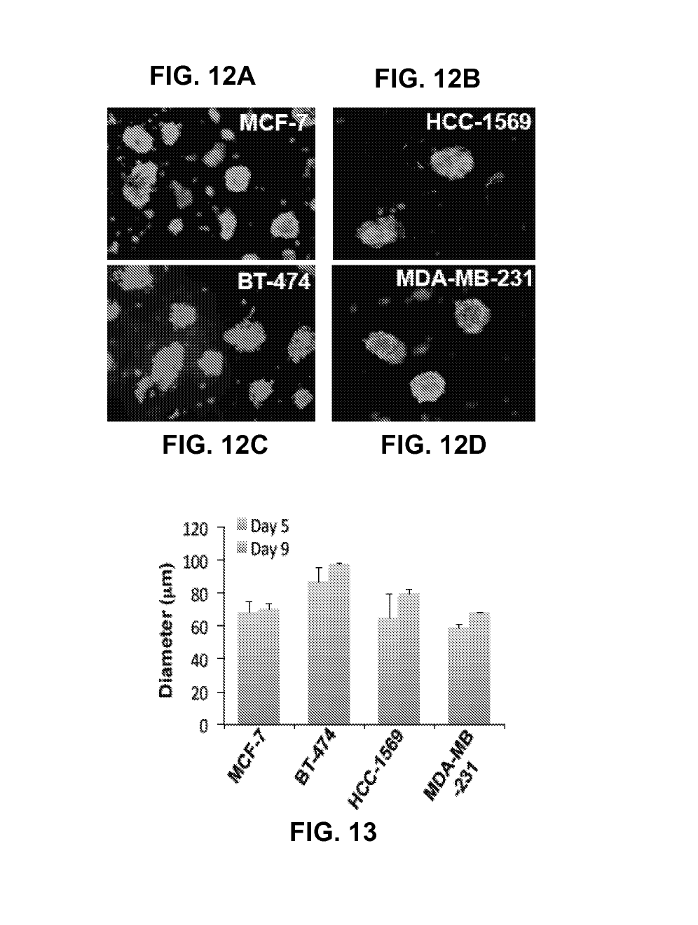

Establishment of tumoroid cultures: Conditions were optimized for tumoroid development. All breast tumor cell lines, MCF7, BT-474 and MDA-MB-231 but HCC1569 form tumoroids with seeding density, 3,000 to 10,000 cells/per 96 well, which is referred to herein as single cell tumoroids (SCTs). This latter cell line was observed to be loosely adherent and had about 50% of cells remain as floaters when cultured on a monolayer. While optimizing tumoroid formation with this cell line, it was unexpectedly observed that HCC-1569 cells readily develop tumoroids in the same frequency as the other cell lines when the culture media is supplemented with 10% matrigel. FIGS. 12A-12D show that all 4 cells lines developed SCTs readily at day 5. Tumoroids growth was monitored for up to day 9 and the size of SCTs at day 5 and day 9 was measured (FIG. 13). Tumoroids of each cell type grew in size by 3-20% differentially.

### Example 8

### Establishment and Characterization of Tumoroid Co-Culture

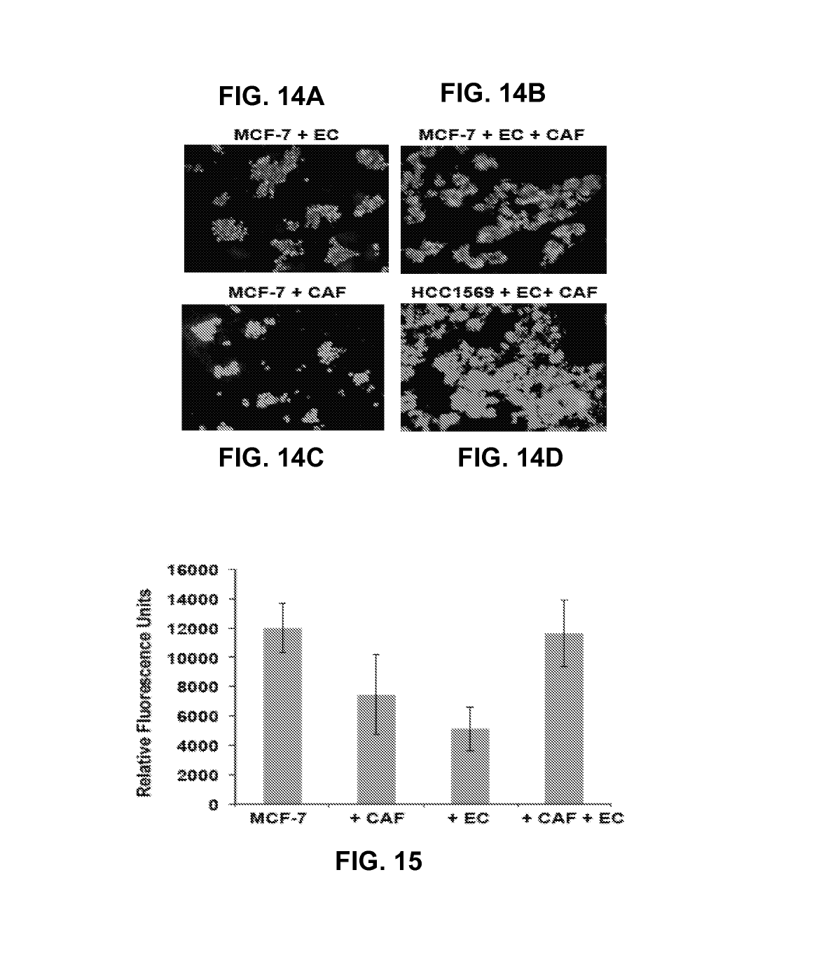

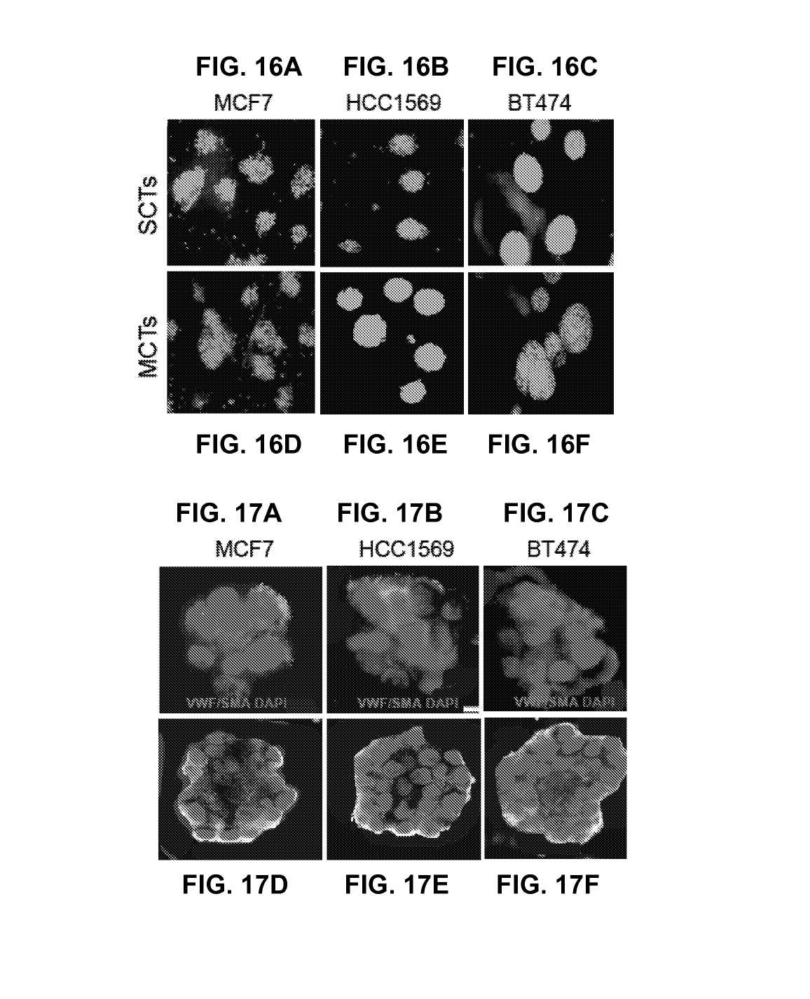

To examine whether co-culture of breast cancer cells with stromal cells, such as CAFs and ECs will form multi-cell tumoroids (MCTs) that can mimic in vivo tumors, co-culture studies were performed. Co-culture of day 5 MCF-7 tumoroids with either ECs or CAFs induced discernable MCTs 3-5 days after co-culture (FIGS. 14A-14D) but net growth of cells reduced (FIG. 15). However, co-culture of MCF-7 cells with both ECs and CAFs not only significantly increased tumoroid size and numbers (FIGS. 14A-14D), but also restored cell growth (FIG. 15). Similarly, HCC-1569 cell line showed significant increase in tumoroid size and numbers when co-cultured with CAFs and ECs simultaneously (FIGS. 14A-14D). Co-culture conditions were optimized and it was observed that co-culturing tumor cells (3-5×103) with ECs (103) and CAFs (103) induced robust MCTs with slightly increased growth potential (FIGS. 16A-16F). Presence of CAFs and ECs in the MCTs was confirmed by IHC using anti-smooth muscle actin (SMA) and anti-von Wille brand factor (vWF) antibodies that are specific for CAFs and ECs, respectively followed by confocal microscopy. A representative fluorescent image and merged z-stacked image of MCTs immunostained for CAFs (red, anti-SMA positive) and ECs (green, anti-vWF-positive) was observed. CAFs were found dispersed throughout the tumoroid whereas ECs were found mostly on the edge of the MCT at day 5 after co-culture (FIGS. 17A-17F). In another set of experiments, co-culture of MDA-MB-231 and ECs also showed tumoroid development, but reduced net cell growth. Together, these results show that co cultures of tumoroids with stromal cells increase significantly tumoroid development.

### Example 9

### Comparison of FiSS Culture with Matrigel-Based 3D Culture

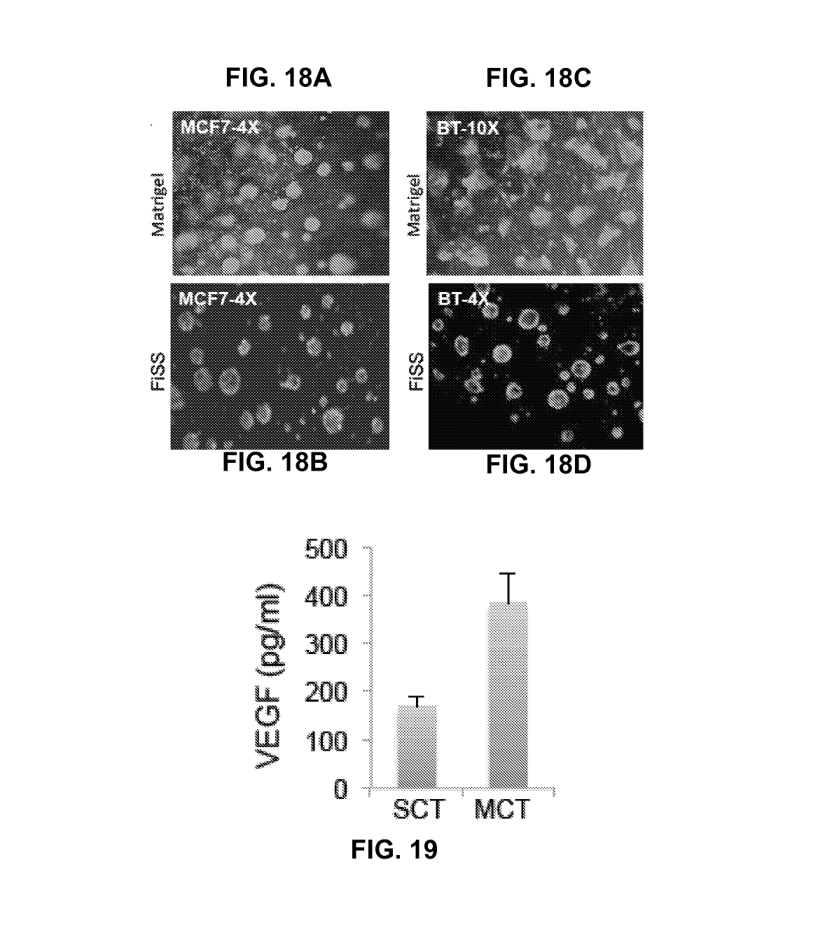

To compare the performance of FiSS tumoroids with other 3D-based culture platforms, spheroid formation in two breast cancer cell lines, MCF7 and BT474 using growth factor reduced matrigel was assessed. For comparison, same number of cells were plated on FiSS and examined. Cells were cultured for 5 days and spheroids were stained with calcein AM and examined by fluorescent microscope. Results presented in FIGS. 18A-18D show that in MCF7 (FIGS. 18A-18B), matrigel culture induced similar number and size of spheroids as in FiSS, whereas in BT474 (FIGS. 18C-18D), matrigel culture formed more disorganized colonies than in FiSS. However, In addition, few colonies in matrigel culture were found to have embedded inside matrigel.

### Example 10

### Evaluation of Cell-Cell or Cell-ECM Adhesion in Tumoroids

Towards characterizing biomarkers in SCTs and MCTs culture, factors that are secreted in the culture supernatants of tumoroids were examined. A comparison of VGEF levels in the supernatant of tumoroids showed that the MCF7-SCTs had less VGEF compared to MCF7-MCTs (FIG. 19). Moreover, as shown in FIGS. 11A-11C, both the BT474-SCTs and -MCTs produced significant amounts of VEGF and IL-6. Interestingly, the co-cultures showed a 2 fold increases in the amounts of VGEF and IL-6 released to the culture medium. This increase in the levels of VGEF and IL-6 produced in co-cultures suggest that they may be critical to increased proliferation of cells and growth of tumoroids seen in co-cultures and therefore these two proteins may serve as markers of clinical efficacy.

Additionally, to characterize factors and molecules released in SCTs and MCTs culture, a human Quantibody Array (RayBiotech Inc.), which utilizes a multiplexed sandwich ELISA assay and enables detection of multiple proteins/factors simultaneously, was used. FIG. 20 shows a can data showing signal intensity of Array 1 and 2; panels 1-8. A human bone metabolism array containing several factors listed in Table 2 of FIG. 21 was chosen. This list includes adhesion molecules (E-selectin, ICAM-1, P-Cadherin, VE-cadherin), \growth factors (aFGF, activin A, androgen receptor (AR), bFGF, bone morphogenic protein (BMP)-2, BMP-4, BMP-6, BMP-7, BMP-9, dickkopf-1 (DKK-1), IGF-1, osteoprotegerin (OPG), osteopontin (OPN), PDGF-BB, TGFβ1, TGFβ2, TGFβ3), chemokines (monocyte chemotactic protein 1 (MCP-1)), macrophage inflammatory protein (MIP)-1α, VCAM-1), cytokines (IL-1α, IL-1β, IL-6, IL-8, IL-11, IL-17, M-CSF), receptor activator of NFkB (RANK), osteoactivin, SDF-1α, TNF related activation induced cytokine (TRANCE)) and matrix metalloproteinases (MMPs), such as MMP-2, -3, -9, -13.

To determine whether any of these factors are expressed in SCTs and MCTs, we incubated QAH-BMA-1000 array with a pool of culture supernatants of BT474-SCTs and MCTs in quadruplets. Experiments were conducted using manufacturer's protocol. An appropriate positive control was used to normalize the signal intensity. Results of the raw data are shown in FIG. 20. Analysis of this data showed that seven of forty-one factors were found significantly altered in MCTs compared to SCTs. These include, growth factors, such as PDGF-BB, OPG and DKK-1, chemokine such as MCP-1, IL-6 and IL-8, and protease MMP-3 (see e.g. FIGS. 23A-25.)

### Example 11

### Determining Growth Factors in Lapatinib Treated Tumoroids Using ELISA

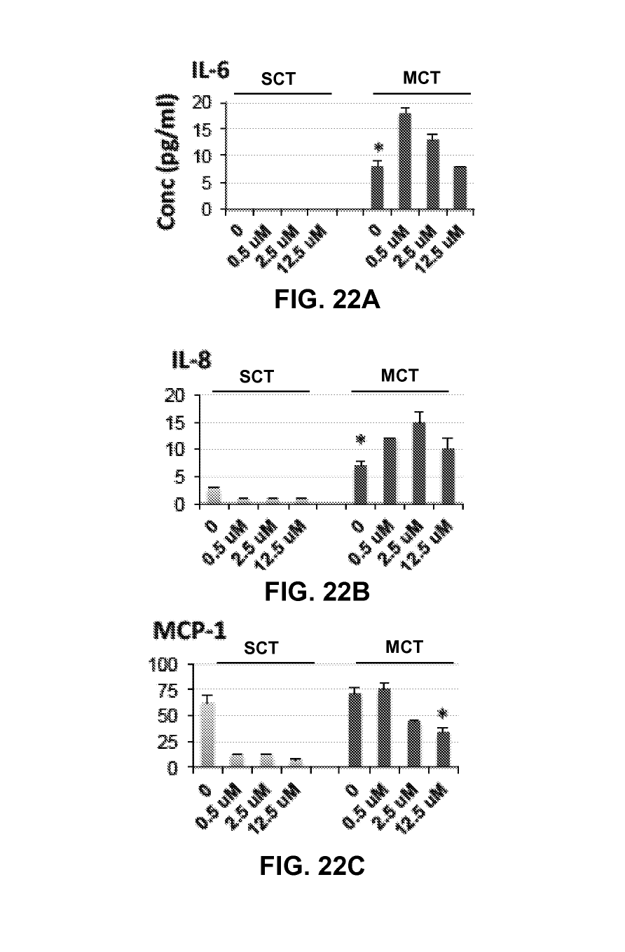

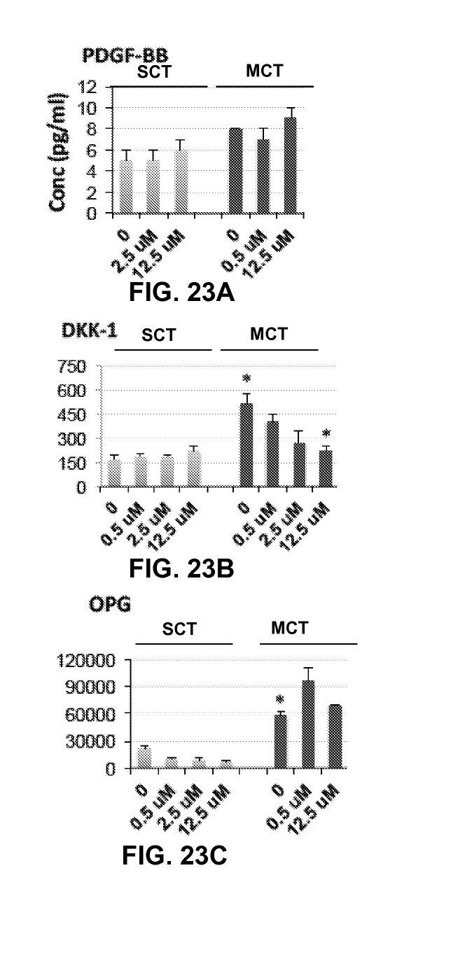

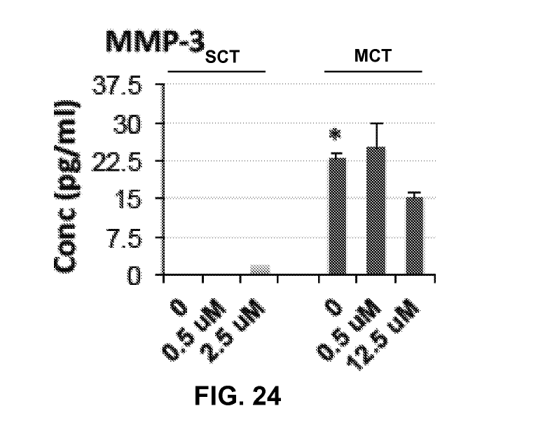

To further validate these results, culture supernatants of SCTs and MCTs using quantibody Array, as described in FIGS. 20-21, were examined. Lapatinib treated cultures were examined for factors that are found differentially expressed in MCTs, compared to SCTs. FIGS. 22A-24 show graphs demonstrating the results. Results showed that culture supernatants of MCTs showed >7 fold increase in IL-6 and IL-8, which were reduced to basal level upon Lapatinib treatment in a dose dependent manner (FIGS. 22A-22B). In contrast, monocyte chemoattractant protein (MCP-1) expression remained unchanged in MCTs; however, Lapatinib treatment abolished MCP-1 expression completely in SCTs, but moderately (at best 50% in the presence of 12.5 uM Lapatinib) in MCTs, suggesting that resistance to Lapatinib in MCTs could be due to sustained MCP-1 (FIG. 22C).

Among growth factors, PDGF-BB (FIG. 23A) was found expressed in both SCTs and MCTs, but Lapatinib treatment did not alter its expression in any tumoroids. In contrast, expression DKK-1 (FIG. 23B), a Wnt signaling inhibitor, and OPG (FIG. 23C), a negative regulator of bone remodeling were found only expressed in MCTs but not in SCTs. Only expression of DKK-1 reduced significantly by Lapatinib treatment (FIG. 23B). It was also found that expression of MMP-3 but not other MMPs was found increased >20 fold in MCTs compared to SCTs and Lapatinib treatment reduced only marginally (FIG. 24). FIG. 25 shows a table that describes clinical relevance of these markers found in MCTs in patients with breast cancer.

### Example 12

### Comparison of 3D FiSS with Matrigel for Predicting Drug Response

To benchmark FiSS-tumoroids against Matrigel-based 3D-culture for prediction of clinical efficacy, 3 d old BT-474 SCTs (cultured on Matrigel or FiSS) were treated with Lapatinib and examined tumoroid formation (FIGS. 26A-26D) and cell viability (FIGS. 27A-27B). Although Matrigel induced numerous smaller size tumoroids compared to FiSS tumoroids, their response to Lapatinib was similar as shown in FIGS. 27A-27B.

### Example 13

### Determination of the Feasibility of the FiSS Platform for Use with High-Content Screening of Samples

### Automated Imaging and Quantification of Tumoroid Cultures

High-content analysis (HCA) is an automated platform for performing fluorescence microscopy and quantitative image analysis, which has been used to analyze cells that had been fixed and stained in a microtiter plate and can quantify (by software) a number of cellular changes, including the phosphorylation, translocation, abundance of a protein on a per cell basis and cytological changes. Data acquisition of FiSS tumoroids in Operetta (Perkin Elmer) to perform HCA analysis was initiated.

To optimize quantification of high content imaging for BT474 tumoroids were incubated for 72 h and then imaged on the Operetta HCI System with a 20× objective. Single plane z-stacked images of BT474 tumoroids on scaffolds were stained with DAPI and images were acquired using the Operetta high content imaging system from Perkin Elmer. Representative fields were selected at 20× magnification and z-stacks of these fields were acquired with an interval of 2 um between each z-plane. Z-stacks were analyzed using the image processing software ImageJ (NIH). The results are shown in FIGS. 28A-28D, which demonstrate that image J analysis can be used to quantify z stacked image data of tumoroids acquired using Operetta (Perkin Elmer). Using HCA we have quantified changes in Ki-67 expression in BT474 tumoroids treated with different concentrations of Lapatinib. Results show dose-dependent Ki-67 expression in Lapatinib treated BT474 tumoroid cultures (FIG. 29).

### Optimizing a Tumoroid Assay: (Z Factor Analysis)

A major stumbling block in 3D cell cultures has been reproducibility, which is important for its incorporation into high-throughput system screening (HTS). In previous studies, the feasibility of PrestoBlue assay was demonstrated, which allows real-time monitoring of cell metabolism and viability through conversion of resazurin (blue) to resorufin (highly fluorescent red). Conversion is proportional to the number of metabolically active cells and therefore can be measured quantitatively. To demonstrate the precision of the PrestoBlue assay (Life Technologies, NY) for LLC1 tumoroids, LLC1 cells were cultured in a 96 well plate preloaded with FiSS. To account for background fluorescence, some wells contained FiSS and media only, without LLC1 tumoroids. After 72 hours, the PrestoBlue assay was performed. The Z′-factor was calculated to evaluate the suitability of the assay for screening applications, which was calculated at 0.63 and 0.72, and considered excellent for HTS readiness (FIGS. 30A-30B). The results demonstrate that in a 96 well plate, the PrestoBlue assay shows minimal well-to-well variation in the tumoroid culture with standard deviation (SD) in replicate experiments found to be within 12% and 10%.

Since viability assays require multiple hour incubation, HTS prefers the ATP-based assay, e.g. CellTiter-Glo (Promega, MD) where the addition of assay reagent immediately ruptures the cells, thereby removing the requirement of incubation of reagent with a viable cell population. To discriminate whether cell proliferation inhibition is due to cytotoxicity or cytostatic effects of the added compounds, cell death relevant parameters of dead cells are measured, e.g. changes in membrane integrity (binding of dye to DNA), caspase-3/7 activity (late stage apoptosis), protease activity. To test the feasibility of ATP based assays for 3D tumoroid cultures, the potential of CellTiter-Glo 2.0, a luminescent viability assay that uses the luciferase reaction to measure ATP, a global indicator of cellular metabolism, was examined. Luciferase, in the presence of Mg2+ and ATP, converts luciferin into oxyluciferin and concomitantly releases energy in the form of luminescence. Signal strength is directly proportional to the amount of ATP present, which correlates with the metabolic activity. This assay can be ideal for HTS, since data can be recorded as soon as 10 minutes after adding reagent, and the luminescent signal is very stable (half-life >5 hours). In a pilot study, we evaluated whether CellTiter-Glo reagent can penetrate tumoroids. Addition of CellTox Green (2×) to media of day 5 BT474-SCTs followed by incubation with luciferin reagent for 10 and 20 min showed that CellTiter-Glo reagent penetrated SCTs (dia: 180 uM) within 20 min of tumoroid lysis (FIG. 31).

A CellTiter-Glo assay was used to evaluate the potential of six compounds in inhibiting proliferation of tumoroids derived from BT474 breast cancer cell line. Results from this pilot study showed, for example, D4 compound to inhibit (>70%) BT474 proliferation at 1:1000 but not at 1:10,000 dose (FIG. 32).

### Automation for Large Volume Production of FiSS

In an effort to increase quality of large-scale production of scaffolds, an adapted Spraybase system was used, which is the world's first integrated instrument that enables electrospinning technology and is compatible with a diverse range of polymers, chemicals and biologics. Spraybase provides ease of use, safety, flexibility and scalability required for FiSS technology. It can be used to form fibers of varying size depending on our need. Spraybase combined with roller drum provides the best approach to produce large volume of FiSS mats for tumoroid technology. Efforts have also been expended to develop further automation of this process that can produce both 96 and 384 microwell FiSS plates for high throughput screening of anti-cancer drugs and high content imaging. The potential of a fabricated 96 well FiSS microplate was used to examine tumoroid formation and the Z′-factor was calculated. The Z-factor was used to evaluate the suitability of the plate for screening applications. Results showed that MCF7 and BT474 cells cultured on the fabricated 96-well microplate formed tumoroids with Z′-factor, 0.87 and 0.846, and Z-factor, 0.812 and 0.501, respectively. These results demonstrate that FiSS-fabricated 96 well microplate fabricated shows minimal well-to-well variation in the tumoroid culture and thus was considered excellent for HTS of anticancer drugs (FIGS. 33A-33B).

We claim:

1. A method of forming multi-cellular tumoroids, the method comprising:
co-culturing tumor cells (TCs), cancer associated fibroblast cells (CAFs), and epithelial cells (ECs) in a cell culture media, where the ratio of the number of TCs to CAFs to ECs is 5 to 1 to 1, and where the cell culture media comprises an amount of a mesenchymal stem cell conditioned media.

2. The method of claim 1, where the tumor cells are breast cancer cells.

3. The method of claim 2, wherein the breast cancer cells are derived from a subject's tumor.

4. The method of claim 1, wherein the mesenchymal stem cell conditioned media is present at least at about 20% of the total amount of the cell culture media.

5. The method of claim 1, wherein the mesenchymal stem cell conditioned media is present at least at about 20% to about 50% of the total amount of the cell culture media.

6.-11. (canceled)

12. A method of determining the efficacy of an anti-cancer drug comprising:
forming a multicellular tumoroid, where forming the multicellular tumoroid comprises:
co-culturing tumor cells (TCs), cancer associated fibroblast cells (CAFs), and epithelial cells (ECs) in a cell culture media, where the ratio of the number of TCs to CAFs to ECs is 5 to 1 to 1, and where the cell culture media comprises an amount of a mesenchymal stem cell conditioned media; and
exposing the multicellular tumoroid to an amount of the anti-cancer drug.

13. The method of claim 12, where the tumor cells are breast cancer cells.

14. The method of claim 13, where the breast cancer cells are derived from a subject's tumor.

15. The method of claim 13, where the breast cancer cells from a standard breast cancer cell line.

16. The method of claim 12, wherein the mesenchymal stem cell conditioned media is present at least at about 20% of the total amount of the cell culture media.

17. The method of claim 12, wherein the mesenchymal stem cell conditioned media is present at least at about 20% to about 50% of the total amount of the cell culture media.

18. The method of claim 12, wherein the mesenchymal stem cell conditioned media contains at least about 800 pg/mL VEGF, at least about 100 pg/mL IL-6, and at least about 1200 pg/mL TGF-β1.

19.-20. (canceled)
